(19) United States
(12) Patent Application Publication
(10) Pub. No.: US 2025/0313568 A1
(43) Pub. Date: Oct. 9, 2025

(54) Organic Compound, Mixture Thereof, And Method For Synthesizing Organic Compound

(71) Applicant: Semiconductor Energy Laboratory Co., Ltd., Kanagawa-ken (JP)

(72) Inventors: Sachiko KAWAKAMI, Atsugi (JP); Tomohiro KUBOTA, Atsugi (JP)

(73) Assignee: Semiconductor Energy Laboratory Co., Ltd., Kanagawa-ken (JP)

(21) Appl. No.: 19/092,165

(22) Filed: Mar. 27, 2025

(30) Foreign Application Priority Data
Apr. 4, 2024 (JP) 2024-060825

(51) Int. Cl. C07D 491/048; H10K 50/11

(52) U.S. Cl. CPC C07D 491/048; H10K 50/11

(57) ABSTRACT

A method for synthesizing an organic compound containing deuterium is provided. In General Formula (G1), Y represents oxygen or sulfur. Each of R¹¹ to R¹⁴ independently represents any one of hydrogen including deuterium, a hydroxyl group, an organoboron group, a boronic acid, an organotin group, and a halogen. Each of X¹ to X⁴ independently represents carbon or nitrogen and any one or two of X¹ to X⁴ represent nitrogen. Each of R¹⁵ to R¹⁸ independently represents hydrogen including deuterium, a hydroxyl group, an organoboron group, a boronic acid, an organotin group, or a halogen. In the case where X¹ to X⁴ each represent nitrogen, R¹⁵ to R¹⁸ bonded to the nitrogen each represent a vacancy. At least one of R¹¹ to R¹⁸ represents deuterium.

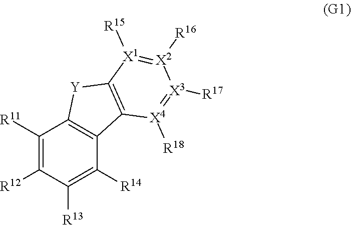

### BACKGROUND OF THE INVENTION

### 1. Field of the Invention

One embodiment of the present invention relates to a method for synthesizing an organic compound. One embodiment of the present invention further relates to a light-emitting device, a light-emitting apparatus, an electronic device, and a lighting device. Note that one embodiment of the present invention is not limited to the above technical field. That is, one embodiment of the present invention relates to an object, a method, a manufacturing method, or a driving method. One embodiment of the present invention relates to a process, a machine, manufacture, or a composition of matter. Specific examples include a semiconductor device, a display apparatus, a liquid crystal display apparatus, and the like.

### 2. Description of the Related Art

A light-emitting device including an EL layer between a pair of electrodes (also referred to as an organic EL element) has characteristics such as thinness, light weight, high-speed response to input signals, and low power consumption; thus, a display including such a light-emitting device is highly useful as a flat panel display or the like.

Displays or lighting devices including light-emitting devices can be suitably used for a variety of electronic devices, and research and development of light-emitting devices has progressed for higher efficiency or longer lifetimes.

Although the characteristics of light-emitting devices have been improved considerably, advanced requirements for various characteristics including efficiency and durability are not yet satisfied. In particular, to solve a problem such as burn-in that is a problem peculiar to EL, it is preferable to inhibit a reduction in efficiency due to deterioration as much as possible.

Deterioration largely depends on an emission center substance and its surrounding materials; therefore, host materials having good characteristics have been actively developed.

For example, Patent Document 1 discloses a technique for substituting a deuterium atom for a hydrogen atom contained in a host material (a deuteration technique). Deuteration of a host material is effective in increasing the lifetime of a light-emitting device, but complicates the synthesis pathway and greatly increases the cost of raw materials. Another problem is that high temperature and high pressure are needed for the synthesis, for example. Another problem is that a purification step after a synthesis reaction is complicated, which makes it difficult to refine an organic compound in which deuteration is performed and obtain high purity.

### REFERENCE

### Patent Document

[Patent Document 1] Japanese Translation of PCT International Application No. 2013-503860

### SUMMARY OF THE INVENTION

An object of one embodiment of the present invention is to provide a method for deuterating an organic compound. Another object of one embodiment of the present invention is to provide a method for synthesizing an organic compound by which part of the organic compound is deuterated. Another object of one embodiment of the present invention is to provide an organic compound that can have an effect of increasing the lifetime of a light-emitting device, by a method for synthesizing an organic compound by which part of the organic compound is selectively deuterated. Another object of one embodiment of the present invention is to make a molecular design with which the degree of complexity of a synthesis pathway can be lowered and increases in temperature, pressure, and the like in a synthesis condition can be suppressed, and to synthesize an organic compound with such a molecular design.

Another object of one embodiment of the present invention is to provide an organic compound that is easy to synthesize. Another object of one embodiment of the present invention is to provide a method for synthesizing a novel organic compound. Another object of one embodiment of the present invention is to provide a novel organic compound. Another object of one embodiment of the present invention is to provide an organic compound that is stable in an excited state. Another object of one embodiment of the present invention is to provide an organic compound that can be used as a host material in which a light-emitting substance is dispersed. Another object of one embodiment of the present invention is to provide a light-emitting device with a long driving lifetime. Another object of one embodiment of the present invention is to provide a novel light-emitting device. Another object of one embodiment of the present invention is to reduce the manufacturing cost of a light-emitting device. Another object of one embodiment of the present invention is to provide a light-emitting apparatus, an electronic device, or a lighting device having low power consumption.

Note that the description of these objects does not preclude the existence of other objects. In one embodiment of the present invention, there is no need to achieve all of these objects. Other objects will be apparent from and can be derived from the description of the specification, the drawings, the claims, and the like.

Note that one embodiment of the present invention does not necessarily achieve all of these objects. Other objects will be apparent from and can be derived from the description of the specification, the drawings, the claims, and the like.

One embodiment of the present invention is a method for synthesizing an organic compound by which an organic compound containing deuterium is obtained.

That is, one embodiment of the present invention is a method for synthesizing an organic compound represented by General Formula (G1) by causing a reaction between an organic compound represented by General Formula (G0) and a transition metal catalyst, heavy water, and a hydrogen molecule.

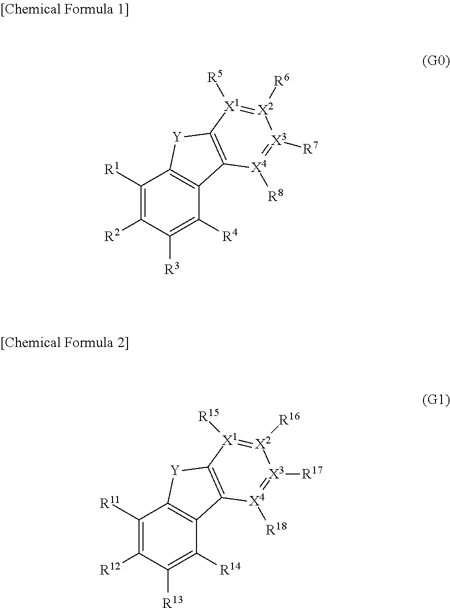

Note that in General Formula (G0) and General Formula (G1) above, Y represents oxygen or sulfur. Each of R¹ to R⁴ and R¹¹ to R¹⁴ independently represents any one of hydrogen (including deuterium), a hydroxyl group, an organoboron group, a boronic acid, an organotin group, and a halogen. Each of X¹ to X⁴ independently represents carbon or nitrogen and any one or two of X¹ to X⁴ represent nitrogen. Each of R⁵ to R⁸ and R¹⁵ to R¹⁸ independently represents hydrogen (including deuterium), a hydroxyl group, an organoboron group, a boronic acid, an organotin group, or a halogen. In the case where X¹ to X⁴ each represent nitrogen, R⁵ to R⁸ and R¹⁵ to R¹⁸ bonded to the nitrogen each represent a vacancy. A sum of deuterium atoms substituting for R¹¹ to R¹⁸ is greater than a sum of deuterium atoms substituting for R¹ to R⁸.

Another embodiment of the present invention is a method for synthesizing an organic compound represented by General Formula (G1) by causing a reaction between an organic compound represented by General Formula (G0) and a transition metal catalyst, heavy water, and an H₂ generation source.

In the above embodiment, the H₂ generation source is formic acid, ammonia borane, or methanol. Alternatively, the H₂ generation source is 2-propanol.

In the above embodiment, the transition metal catalyst contains a platinum group element.

Another embodiment of the present invention is an organic compound represented by General Formula (G1).

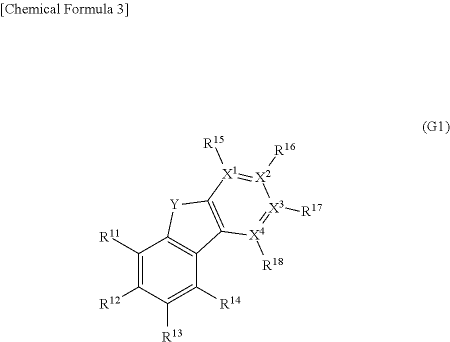

In General Formula (G1) above, each of R¹¹ to R¹⁴ independently represents any one of hydrogen (including deuterium), a hydroxyl group, and a halogen. Each of X¹ to X⁴ independently represents carbon or nitrogen and any one or two of X¹ to X⁴ represent nitrogen. Each of R¹⁵ to R¹⁸ independently represents hydrogen (including deuterium), a hydroxyl group, or a halogen. In the case where X¹ to X⁴ each represent nitrogen, R¹⁵ to R¹⁸ bonded to the nitrogen each represent a vacancy. Y represents oxygen or sulfur. At least one of R¹¹ to R¹⁸ represents deuterium.

Another embodiment of the present invention is an organic compound represented by Structural Formula (100).

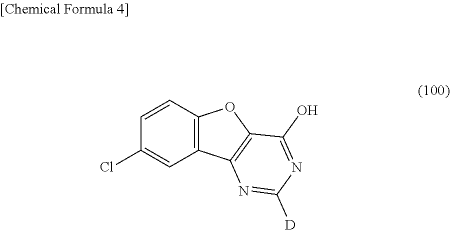 [SMILES: [2H]c1nc(O)c2oc3ccc(Cl)cc3c2n1]

Another embodiment of the present invention is an organic compound represented by Structural Formula (100), an organic compound represented by Structural Formula (100-1), or a mixture thereof.

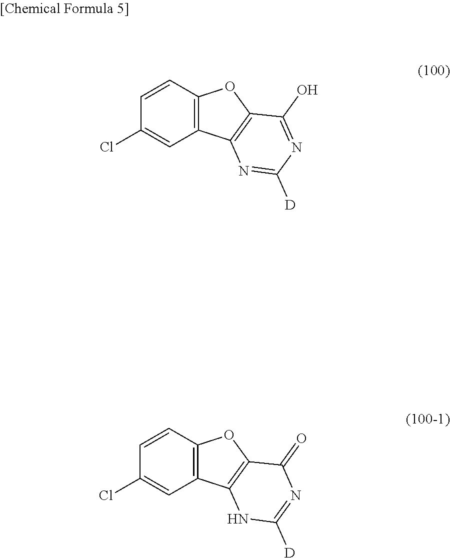 [SMILES: [2H]c1nc(O)c2oc3ccc(Cl)cc3c2n1 ; [2H]c1nc(=O)c2oc3ccc(Cl)cc3c2[nH]1]

Another embodiment of the present invention is an electronic device including a sensor, an operation button, a speaker, or a microphone, and a light-emitting device or a light-receiving device including the above organic compound.

Another embodiment of the present invention is a lighting device including a housing and the light-emitting device or a light-receiving device including the above organic compound.

According to one embodiment of the present invention, a method for synthesizing an organic compound can be provided. According to another embodiment of the present invention, a method for synthesizing an organic compound by which part of the organic compound is deuterated can be provided. According to another embodiment of the present invention, an organic compound that can have an effect of increasing the lifetime of a light-emitting device can be provided by a method for synthesizing an organic compound by which part of the organic compound is selectively deuterated. As a result, the degree of complexity of a synthesis pathway, the equivalent or kind of a reagent used, and the temperature, pressure, and the like in the synthesis pathway can be reduced in substitution of deuterium atoms for some or all of hydrogen atoms of an organic compound.

According to another embodiment of the present invention, an organic compound that is easy to synthesize can be provided. According to another embodiment of the present invention, a method for synthesizing a novel organic compound can be provided. According to another embodiment of the present invention, a novel organic compound can be provided. According to another embodiment of the present invention, a method for synthesizing a stable organic compound that is less likely to react in an excited state can be provided. According to another embodiment of the present invention, an organic compound that can be used for a light-emitting device can be provided. According to another embodiment of the present invention, an organic compound that can be used for a light-emitting layer of a light-emitting device can be provided. According to another embodiment of the present invention, an organic compound that can be used for a carrier-transport layer of a light-emitting device can be provided. According to another embodiment of the present invention, a novel light-emitting device can be provided. According to another embodiment of the present invention, a light-emitting device with a long driving lifetime can be provided. According to another embodiment of the present invention, the manufacturing cost of a light-emitting device can be reduced. According to another embodiment of the present invention, a light-emitting apparatus, an electronic device, or a lighting device having low power consumption can be provided.

Note that the description of these effects does not preclude the existence of other effects. One embodiment of the present invention does not necessarily have all these effects. Other effects will be apparent from and can be derived from the description of the specification, the drawings, the claims, and the like.

### BRIEF DESCRIPTION OF THE DRAWINGS

In the accompanying drawings:

FIGS. 1A and 1B illustrate light-emitting devices;

FIGS. 2A and 2B illustrate light-emitting devices;

FIGS. 3A and 3B illustrate light-emitting devices;

FIGS. 4A and 4B are perspective views illustrating a structure example of a display module;

FIGS. 5A and 5B are cross-sectional views illustrating structure examples of a display apparatus;

FIG. 6 is a perspective view illustrating a structure example of a display apparatus;

FIG. 7 is a cross-sectional view illustrating a structure example of a display apparatus;

FIG. 8 is a cross-sectional view illustrating a structure example of a display apparatus;

FIGS. 9A to 9C are a cross-sectional view and top views illustrating a structure example of a display apparatus;

FIG. 10 is a cross-sectional view illustrating a structure example of a display apparatus.

FIGS. 11A to 11C are a cross-sectional view and top views illustrating a structure example of a display apparatus;

FIG. 12 shows a ¹H-NMR spectrum of an organic compound;

FIG. 13 shows a ¹³C-NMR spectrum of an organic compound; and

FIG. 14 shows an MS spectrum of an organic compound.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Therefore, the present invention should not be construed as being limited to the description in the following embodiments.

Note that the position, size, range, or the like of each component illustrated in drawings and the like is not accurately represented in some cases for easy understanding. Therefore, the disclosed invention is not necessarily limited to the position, size, range, or the like disclosed in the drawings and the like.

In describing structures of the invention with reference to the drawings in this specification and the like, the same components in different drawings are commonly denoted by the same reference numeral.

### Embodiment 1

In this embodiment, a method for synthesizing an organic compound of one embodiment of the present invention is described. The organic compound of one embodiment of the present invention is represented by General Formula (G1) below.

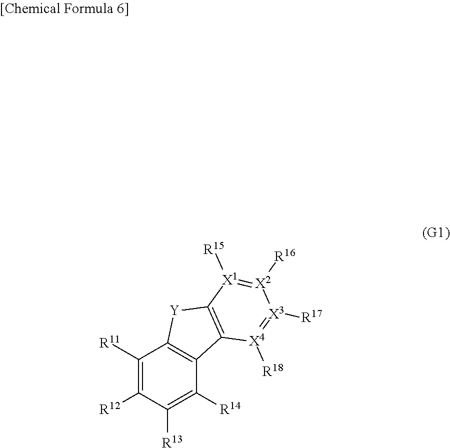

In General Formula (G1) above, each of R¹¹ to R¹⁴ independently represents any one of hydrogen (including deuterium), a hydroxyl group, and a halogen. Each of X¹ to X⁴ independently represents carbon or nitrogen, and any one or two of X¹ to X⁴ represent nitrogen. Each of R¹⁵ to R¹⁸ independently represents hydrogen (including deuterium), a hydroxyl group, or a halogen. In the case where X¹ to X⁴ each represent nitrogen, R¹⁵ to R¹⁸ bonded to the nitrogen each represent a vacancy. In the case where R¹⁵ to R¹⁸ each represent a hydroxyl group, the hydroxyl group may become a ketone derivative that is a tautomer of the hydroxyl group. Note that Y represents oxygen or sulfur. At least one of R¹¹ to R¹⁸ represents deuterium.

An example of a method for synthesizing the organic compound of one embodiment of the present invention represented by General Formula (G1) above will be described below.

As shown in Synthesis Scheme (s-1) below, the organic compound represented by General Formula (G1) above can be synthesized by a reaction between an organic compound represented by General Formula (G0), a transition metal catalyst M, heavy water (D₂O), and a hydrogen molecule (H₂).

Note that the heavy water (D₂O) that can be used in Synthesis Scheme (s-1) below may include water (H₂O) and semi-heavy water (DHO). In the case where H₂O and DHO are included, the purity of D₂O is preferably higher than or equal to 99%. The hydrogen molecule (H₂) may include a deuterium molecule (D₂) or a hydrogen deuterium molecule (DH).

Alternatively, in Synthesis Scheme (s-1) below, an H₂ generation source that can generate a hydrogen molecule in the presence of a transition metal catalyst can be used instead of the hydrogen molecule (H₂). That is, the organic compound represented by General Formula (G1) can be synthesized by a reaction between the H₂ generation source that can generate a hydrogen molecule in the presence of a transition metal catalyst, such as formic acid or alcohol, the organic compound represented by General Formula (G0), the transition metal catalyst M, and the heavy water (D₂O).

As the H₂ generation source that can generate a hydrogen molecule in the presence of a transition metal catalyst, it is possible to use formic acid, ammonia borane, methanol, or 2-propanol, for example. In particular, 2-propanol is preferable because it has neutrality and high solubility in the organic compound represented by General Formula (G0), which is a substrate, or the organic compound represented by General Formula (G1), which is a product, and thus weakly interacts with these organic compounds in a reaction system.

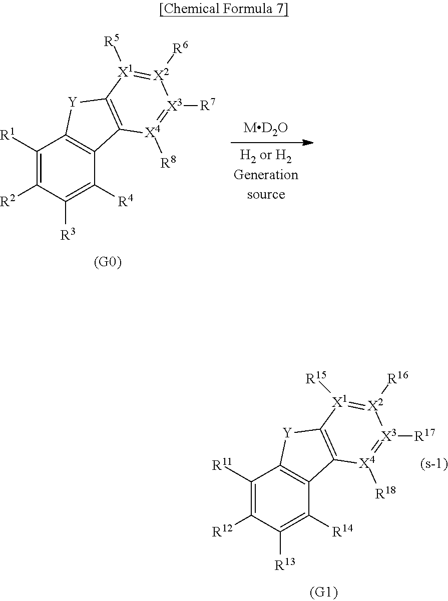

In General Formula (G0) in the above synthesis scheme, each of R¹ to R⁴ independently represents hydrogen (including deuterium), a hydroxyl group, an organoboron group, a boronic acid, an organotin group, or a halogen, and each of R⁵ to R⁸ independently represents hydrogen (including deuterium), a hydroxyl group, an organoboron group, a boronic acid, an organotin group, or a halogen. Furthermore, each of X¹ to X⁴ independently represents carbon or nitrogen, and any one or two of X¹ to X⁴ represent nitrogen. Note that Y represents oxygen or sulfur. That is, the organic compound represented by General Formula (G1) is an organic compound obtained by substituting deuterium for at least one of R¹ to R⁸ in General Formula (G0) (also referred to as a deuterated organic compound).

Thus, the total number (sum) of deuterium atoms in R¹¹ to R¹⁸ is larger than the total number (sum) of deuterium atoms in R¹ to R⁸, and the deuteration rates of R¹¹ to R¹⁸ are higher than or equal to those of R¹ to R⁸, respectively. Specifically, in comparison between R¹ and R¹¹, the deuteration rate of R¹¹ is higher than or equal to that of R¹. Similarly in comparisons between R² and R¹², R³ and R¹³, R⁴ and R¹⁴, R⁵ and R¹⁵, R⁶ and R¹⁶, R⁷ and R¹⁷, and R⁸ and R¹⁸, the deuteration rates of R¹², R¹³, R¹⁴, R¹⁵, R¹⁶, R¹⁷, and R¹⁸ are higher than or equal to those of R², R³, R⁴, R⁵, R⁶, R⁷, and R⁸, respectively, as in the comparison between R¹ and R¹¹.

In the organic compound represented by General Formula (G0) or General Formula (G1) above, each of R¹ to R⁸ and R¹¹ to R¹⁸ preferably represents iodine, bromine, or chlorine when representing a halogen.

In particular, in the case where R¹ to R⁸ and R¹¹ to R¹⁸ each represent a halogen, a halogen-carbon bond in General Formula (G1) is suitable as a substrate for a chemical reaction for forming a carbon-carbon bond, typified by Suzuki coupling. Thus, in the case where a target compound is synthesized using the compound represented by General Formula (G1) as a substrate, R¹ to R⁸ and R¹¹ to R¹⁸ each preferably represent a halogen because a reaction of direct conversion into the target compound can be performed.

In Synthesis Scheme (s-1), the transition metal catalyst M represents a catalyst containing a transition metal element. The transition metal element is preferably a platinum group element such as platinum (Pt), palladium (Pd), rhodium (Rh), iridium (Ir), or ruthenium (Ru), specifically. Furthermore, heavy water (D₂O) is used as a solvent and a deuterium source. For activating the transition metal catalyst M, a hydrogen molecule (H₂) (including a deuterium molecule (D₂) and a hydrogen deuterium molecule (DH)) can be used. Instead of the hydrogen molecule, a compound that generates a hydrogen molecule by reacting with the transition metal catalyst M in a reaction system can also be used.

With the use of the synthesis method of one embodiment of the present invention, an organic compound can be deuterated not through an extremely-high-temperature state at higher than or equal to 200° C. or an extremely-high-pressure state at higher than or equal to 1 MPa. In addition, a stable organic compound whose reaction is less likely to proceed can be easily deuterated. Specifically, in the synthesis method of one embodiment of the present invention, direct conversion into a deuterium compound can be performed under a mild reaction condition with a temperature lower than 200° C. or a pressure lower than 1.0 MPa. Since the organic compound containing deuterium is not synthesized through a multi-step synthesis pathway, the manufacturing cost of a light-emitting device can be reduced.

According to another embodiment of the present invention, a deuterated organic compound that can be used as a material for a light-emitting device can be provided at low cost. For example, in the case where the deuterated organic compound has an electron-transport property, the organic compound can be used for an electron-transport layer, a hole-blocking layer, a host material of a light-emitting layer, or the like.

With the use of a deuterated organic compound as a material for a light-emitting device, the driving lifetime of the light-emitting device can be increased. Accordingly, with the deuterated organic compound, the light-emitting device can have a long driving lifetime, which enables a highly reliable electronic device and contributes to a reduction in the device cost for a consumer. Furthermore, a deuterated light-emitting material tends to have improved emission efficiency, thereby achieving a light-emitting apparatus, an electronic device, or a lighting device with low power consumption.

### SPECIFIC EXAMPLES

Next, specific examples of the organic compound represented by General Formula (G1) above are shown below.

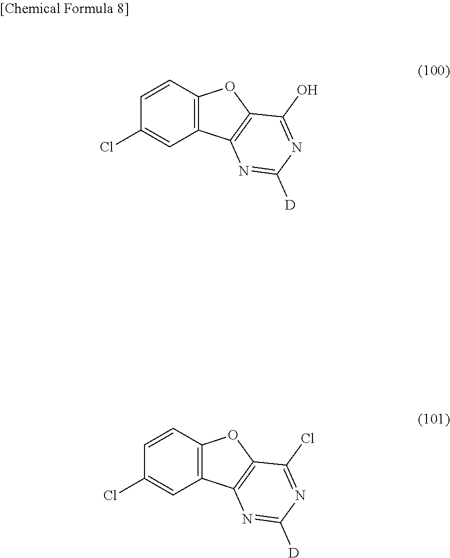

Structural Formulae (100) and (101) above are examples of the organic compound represented by General Formula (G1) above. The organic compound of one embodiment of the present invention is not limited thereto.

Note that the organic compound represented by Structural Formula (100) above and a tautomer represented by Structural Formula (100-1) below exist in an equilibrium state represented by Structural Formula (100-2). Thus, one embodiment of the present invention is a mixture including any of the organic compound represented by Structural Formula (100) and the tautomer represented by Structural Formula (100-1).

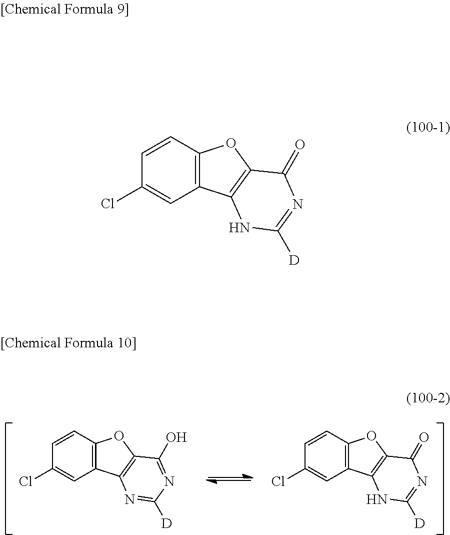

This embodiment can be freely combined with any of the other embodiments and the examples.

### Embodiment 2

In this embodiment, light-emitting devices of one embodiment of the present invention will be described in detail.

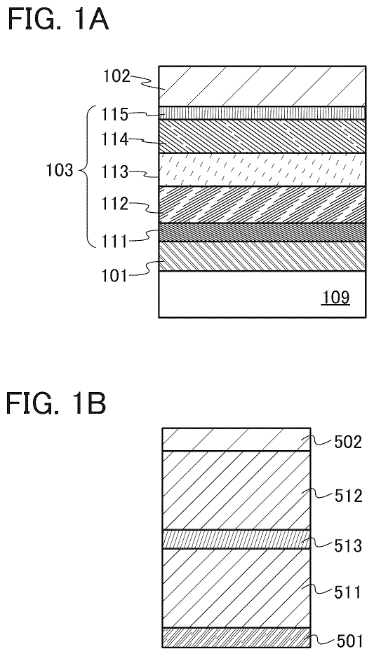

FIGS. 1A and 1B are schematic views of a light-emitting device of one embodiment of the present invention. The light-emitting device includes a first electrode 101 over an insulator 109, and an organic compound layer 103 between the first electrode 101 and a second electrode 102. The organic compound layer 103 includes at least a light-emitting layer 113 and an electron-injection layer 115. The light-emitting layer 113 contains a light-emitting substance and emits light when voltage is applied between the first electrode 101 and the second electrode 102.

The organic compound layer 103 preferably includes, besides the light-emitting layer 113 and the electron-injection layer 115, functional layers such as a hole-injection layer 111, a hole-transport layer 112, and an electron-transport layer 114, as illustrated in FIG. 1A. The organic compound layer 103 may include functional layers other than the above functional layers, such as a hole-blocking layer, an exciton-blocking layer, and an intermediate layer. Alternatively, any of the above-described layers may be omitted.

For the organic compound layer 103, an organic compound that is synthesized by the synthesis method described in Embodiment 1 using a deuterated intermediate can be used. With the use of a deuterated organic compound for the light-emitting device, the lifetime of the light-emitting device can be increased.

This embodiment describes an example where the first electrode 101 includes an anode, the second electrode 102 includes a cathode, and the first electrode 101 is formed on the insulator 109 side; however, a structure where the second electrode 102 is formed on the insulator 109 side, what is called an inversely stacked structure, may be employed. In this case, the light-emitting device has a stacked-layer structure where the second electrode 102, the electron-injection layer 115, (the electron-transport layer 114), the light-emitting layer 113, (the hole-transport layer 112, the hole-injection layer 111), and the first electrode 101 are stacked in this order from the insulator 109 side. In the case of such a light-emitting device having an inversely stacked structure, the relatively stable hole-injection layer 111 serves as a surface; thus, the light-emitting device can have higher reliability.

The first electrode 101 and the second electrode 102 may each have a single-layer structure or a stacked-layer structure. In the case of the stacked-layer structure, a layer in contact with the organic compound layer 103 serves as an anode or a cathode. In the case where the electrodes each have the stacked-layer structure, there is no limitation on work functions of materials for layers other than the layer in contact with the organic compound layer 103, and the materials can be selected in accordance with required properties such as a resistance value, processing easiness, reflectivity, light-transmitting property, and stability.

The anode is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide containing silicon or silicon oxide (ITSO: indium tin silicon oxide), indium oxide-zinc oxide, and indium oxide containing tungsten oxide and zinc oxide (IWZO). Such conductive metal oxide films are usually formed by a sputtering method, but may be formed by application of a sol-gel method or the like. For example, a film of indium oxide-zinc oxide is formed by a sputtering method using a target in which 1 wt % to 20 wt % zinc oxide is added to indium oxide. Furthermore, a film of indium oxide containing tungsten oxide and zinc oxide (IWZO) can be formed by a sputtering method using a target in which 0.5 wt % to 5 wt % tungsten oxide and 0.1 wt % to 1 wt % zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), titanium, (Ti), aluminum (Al), nitride of a metal material (e.g., titanium nitride), or the like can be used for the anode. The anode may be a stack of layers formed of any of these materials. For example, a film in which Al, Ti, and ITSO are stacked in this order over Ti is preferable because the film has high efficiency owing to high reflectivity and enables high resolution of several thousand ppi. Graphene can also be used for the anode. When a composite material that can be included in the hole-injection layer 111, which is described later, is used for a layer (typically, the hole-injection layer) in contact with the anode, an electrode material can be selected regardless of its work function.

The hole-injection layer 111 is provided in contact with the anode and has a function of facilitating injection of holes to the organic compound layer 103. The hole-injection layer 111 can be formed using a phthalocyanine-based compound such as phthalocyanine (abbreviation: H₂Pc), a phthalocyanine-based complex compound such as copper phthalocyanine (abbreviation: CuPc), an aromatic amine compound such as 4,4′-bis[N-(4-diphenylaminophenyl)-N-phenylamino]biphenyl (abbreviation: DPAB) or 4,4′-bis(N-{4-[N-(3-methylphenyl)-N′-phenylamino]phenyl}-N-phenylamino)biphenyl (abbreviation: DNTPD), or a high molecular compound such as poly(3,4-ethylenedioxythiophene)/poly(styrenesulfonic acid) (abbreviation: PEDOT/PSS).

The hole-injection layer 111 may be formed using a substance having an acceptor property. Examples of the substance having an acceptor property include organic compounds having an electron-withdrawing group (a halogen group, a cyano group, or the like), such as 7,7,8,8-tetracyano-2,3,5,6-tetrafluoroquinodimethane (abbreviation: F4-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene) malononitrile. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a significantly high acceptor property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as molybdenum oxide, vanadium oxide, ruthenium oxide, tungsten oxide, or manganese oxide can be used, other than the above-described organic compounds.

The hole-injection layer 111 is preferably formed using a composite material containing any of the aforementioned materials having an acceptor property and an organic compound having a hole-transport property.

As the organic compound having a hole-transport property used in the composite material, any of a variety of organic compounds such as aromatic amine compounds, heteroaromatic compounds, aromatic hydrocarbons, and high molecular compounds (e.g., oligomers, dendrimers, and polymers) can be used. Note that the organic compound having a hole-transport property used in the composite material preferably has a hole mobility higher than or equal to 1×10⁻⁶ cm²/Vs. The organic compound having a hole-transport property used in the composite material preferably includes a condensed aromatic hydrocarbon ring or a π-electron rich heteroaromatic ring. As the condensed aromatic hydrocarbon ring, an anthracene ring, a naphthalene ring, or the like is preferable. As the π-electron rich heteroaromatic ring, a condensed aromatic ring having at least one of a pyrrole skeleton, a furan skeleton, and a thiophene skeleton in the ring is preferable; specifically, a carbazole ring, a dibenzothiophene ring, or a ring in which an aromatic ring or a heteroaromatic ring is further condensed to a carbazole ring or a dibenzothiophene ring is preferable.

The above-described composite material containing a material having an acceptor property and an organic compound having a hole-transport property efficiently enables interaction between materials. Thus, in a film containing the composite material, the spin density attributed to a signal observed at a g-factor of approximately 2.00 is measured by ESR to be, preferably higher than or equal to 1×10¹⁷ spins/cm³.

Such an organic compound having a hole-transport property further preferably has any of a carbazole skeleton, a dibenzofuran skeleton, a dibenzothiophene skeleton, and an anthracene skeleton. In particular, an aromatic amine including a substituent having a dibenzofuran ring or a dibenzothiophene ring, an aromatic monoamine having a naphthalene ring, or an aromatic monoamine in which a 9-fluorenyl group is bonded to nitrogen of an amine through an arylene group may be used. Note that the organic compound having a hole-transport property preferably includes an N,N-bis(4-biphenyl)amino group to enable manufacturing a light-emitting device with a long lifetime.

Specific examples of the organic compound having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAPNβNβ-03), 4,4′-diphenyl-4″-(7-phenyl)naphthyl-2-yl)triphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Examples of the aromatic amine compound that can be used as the organic material having a hole-transport property include N,N′-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N-(3-methylphenyl)-N-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B).

The formation of the hole-injection layer 111 can improve the hole-injection property, which allows the light-emitting device to be driven at a low voltage.

Among substances having an acceptor property, the organic compound having an acceptor property is easy to use because it is easily deposited by vapor deposition.

The hole-transport layer 112 is formed using an organic compound having a hole-transport property. The organic compound having a hole-transport property preferably has a hole mobility of 1×10⁻⁶ cm²/Vs or higher.

Examples of the organic material having a hole-transport property include compounds having an aromatic amine skeleton, such as 4,4′-bis[N-(1-naphthyl)-N-phenylamino]biphenyl (abbreviation: NPB), N,N-diphenyl-N,N′-bis(3-methylphenyl)-4,4′-diaminobiphenyl (abbreviation: TPD), N,N′-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 9,9′-diphenyl-9H,9′H-3,3′-bicarbazole (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-9H,9′H-3,3′-bi-9H-carbazole (abbreviation: βNCCP), 9-(3-biphenyl)-9′-(2-naphthyl)-3,3′-bi-9H-carbazole (abbreviation: βNCCmBP), 9-(4-biphenyl)-9′-(2-naphthyl)-3,3′-bi-9H-carbazole (abbreviation: βNCCBP), 9,9′-di-2-naphthyl-3,3′-9H,9′H-bicarbazole (abbreviation: BisβNCz), 9-(2-naphthyl)-9′-[1,1′:4′,1″-terphenyl]-3-yl-3,3′-9H,9′H-bicarbazole, 9-(2-naphthyl)-9′-[1,1′:3′,1″-terphenyl]-3-yl-3,3′-9H,9′H-bicarbazole, 9-(2-naphthyl)-9′-[1,1′: 3′,1″-terphenyl]-5′-yl-3,3′-9H,9′H-bicarbazole, 9-(2-naphthyl)-9′-[1,1′: 4′,1″-terphenyl]-4-yl-3,3′-9H,9′H-bicarbazole, 9-(2-naphthyl)-9′-[1,1′: 3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, and 9-(triphenylen-2-yl)-9′-[1,1′:3′,1″-terphenyl]-4-yl-3,3′-9H,9′H-bicarbazole; compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. Note that any of the substances given as examples of the organic material having a hole-transport property used for the composite material for the hole-injection layer 111 can also be suitably used as the material contained in the hole-transport layer 112.

The light-emitting layer 113 is a layer containing a light-emitting substance and preferably contains a light-emitting substance and a host material. The light-emitting layer 113 may additionally contain other materials. Alternatively, the light-emitting layer 113 may be a stack of two layers with different compositions.

As the light-emitting substance, fluorescent substances, phosphorescent substances, substances exhibiting thermally activated delayed fluorescence (TADF), or other light-emitting substances may be used.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPrn), N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis(N,N,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N,N-triphenyl-1,4-phenylenediamine (abbreviation: 2DPAPPA), N,N,N′,N′,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-(2-[4-(dimethylamino)phenyl]ethenyl)-6-methyl-4H-pyran-4-ylidene)propanedinitrile (abbreviation: DCM1), 2-(2-methyl-6-[2-(2,3,6,7-tetrahydro-1H,5H-benzo[ij]quinolizin-9-yl)ethenyl]-4H-pyran-4-ylidene)propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-(2-isopropyl-6-[2-(1,1,7,7-tetramethyl-2,3,6,7-tetrahydro-1H,5H-benzo[ij]quinolizin-9-yl)ethenyl]-4H-pyran-4-ylidene)propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis(2-[4-(dimethylamino)phenyl]ethenyl)-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPrn-03), N,N′-diphenyl-N,N-bis(9-phenyl-9H-carbazol-2-yl)naphtho[2,3-b;6,7-b′]bisbenzofuran-3,10-diamine (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, or high reliability.

A condensed heteroaromatic compound containing nitrogen and boron, especially a compound having a diaza-boranaphtho-anthracene skeleton, exhibits a narrow emission spectrum, emits blue light with favorable color purity, and can thus be used suitably. Examples of the compound include 5,9-diphenyl-5,9-diaza-13b-boranaphtho[3,2,1-delanthracene (abbreviation: DABNA1), 9-[(1,1′-diphenyl)-3-yl]-N,N,5,11-tetraphenyl-5,9-dihydro-5,9-diaza-13b-boranaphtho[3,2,1-de]anthracen-3-amine (abbreviation: DABNA2), 2,12-di(tert-butyl)-5,9-di(4-tert-butylphenyl)-N,N-diphenyl-5H,9H-[1,4]benzazaborino[2,3,4-kl]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-kl]phenazaborine (abbreviation: Me-tBu4DABNA), N⁷,N⁷,N¹³,N¹³,5,9,11,15-octaphenyl-5H,9H,11H,15H-[1,4]benzazaborino[2,3,4-kl][1,4]benzazaborino[4′,3′,2′:4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′,2′,1′: 8,1][1,4]benzazaborino[2,3,4-kl]phenazaborine (abbreviation: BBCz-G), 9,11-bis[3,6-bis(1,1-dimethylethyl)-9H-carbazolyl-9-yl]-2,5,15,18-tetrakis(1,1-dimethylethyl)indolo[3,2,1-de]indolo[3′,2′,1′: 8,1][1,4]benzazaborino[2,3,4-kl]phenazaborine (abbreviation: BBCz-Y), or the like can be suitably used.

Examples of the material that can be used when a phosphorescent substance is used as the light-emitting substance in the light-emitting layer are as follows.

The examples include organometallic iridium complexes having a 4H-triazole skeleton, such as tris(2-[5-(2-methylphenyl)-4-(2,6-dimethylphenyl)-4H-1,2,4-triazol-3-yl-κN²]phenyl-KC)iridium(III) (abbreviation: [Ir(mpptz-dmp)₃]) and tris(5-methyl-3,4-diphenyl-4H-1,2,4-triazolato)iridium(II) (abbreviation: [Ir(Mptz)₃]); organometallic iridium complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes having an imidazole skeleton, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-(1-[2,6-bis(1-methylethyl)phenyl]-1H-imidazol-2-yl-κN³)-4-cyanophenyl-κC)iridium(III) (abbreviation: CNImIr); organoiridium complexes having a benzimizazolidene skeleton, such as tris[(6-tert-butyl-3-phenyl-2H-imidazo[4,5-b]pyrazin-1-yl-κC)phenyl-κC]iridium(III) (abbreviation: [Ir(cb)₃]); and organometallic iridium complexes in which a phenylpyridine derivative including an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis(2-[3′,5′-bis(trifluoromethyl)phenyl]pyridinato-N,C^{2′})iridium(III) picolinate (abbreviation: [Ir(CF₃ppy)₂(pic)]), and bis[2-(4′,6′-difluorophenyl)pyridinato-N,C^{2′}-]iridium(III) acetylacetonate (abbreviation: FIracac). These compounds emit blue phosphorescent light and have an emission peak in the wavelength range of 450 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′}-)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN)phenyl-κC]iridium(III) (abbreviation: [Ir(5mppy-d₃)₂(mbfpypy-d₃)]), [2-d₃-methyl-(2-pyridinyl-κN)benzofuro[2,3-b]pyridine-KC]bis[2-(2-pyridinyl-κN)phenyl-κC]iridium(III) (abbreviation: [Ir(ppy)₂(mbfpypy-d₃)]), [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), [2-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]), and [2-(4-methyl-5-phenyl-2-pyridinyl-κN)phenyl-κC]bis[2-(2-pyridinyl-κN)phenyl-κC]iridium(III) (abbreviation: [Ir(ppy)₂(mdppy)]); and rare earth metal complexes such as tris(acetylacetonato) (monophenanthroline)terbium(II) (abbreviation: [Tb(acac)₃(Phen)]). These compounds mainly emit green phosphorescent light and have an emission peak in the wavelength range of 500 nm to 600 nm. Note that organometallic iridium complexes having a pyrimidine skeleton have distinctively high reliability or emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), (3,7-diethyl-4,6-nonanedionato-κO⁴,κO⁶)bis[2,4-dimethyl-6-[7-(1-methylethyl)-1-isoquinolinyl-κN]phenyl-κC]iridium(III), and (3,7-diethyl-4,6-nonanedionato-κO⁴,κO⁶)bis[2,4-dimethyl-6-[5-(1-methylethyl)-2-quinolinyl-κN]phenyl-κC]iridium(III); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescent light and have an emission peak in the wavelength range of 600 nm to 700 nm. Furthermore, the organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

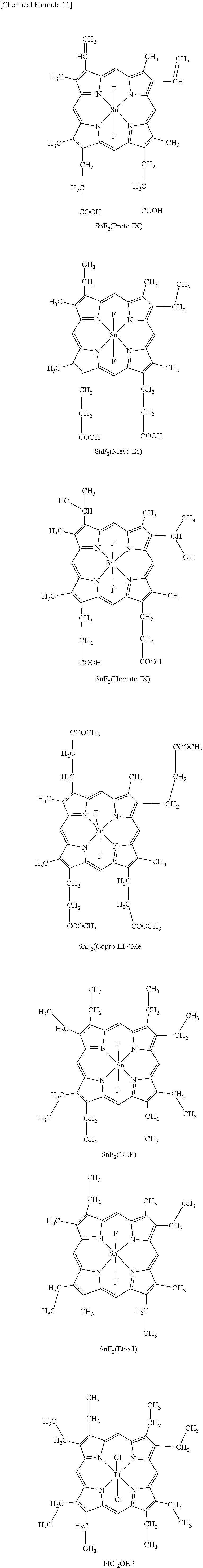

Alternatively, it is possible to use a heterocyclic compound including one or both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of having high electron-transport and hole-transport properties owing to a π-electron rich heteroaromatic ring and a π-electron deficient heteroaromatic ring. Among skeletons including the π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high electron-acceptor properties and high reliability. Among skeletons including the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferable because the electron-donating property of the π-electron rich heteroaromatic ring and the acceptor property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

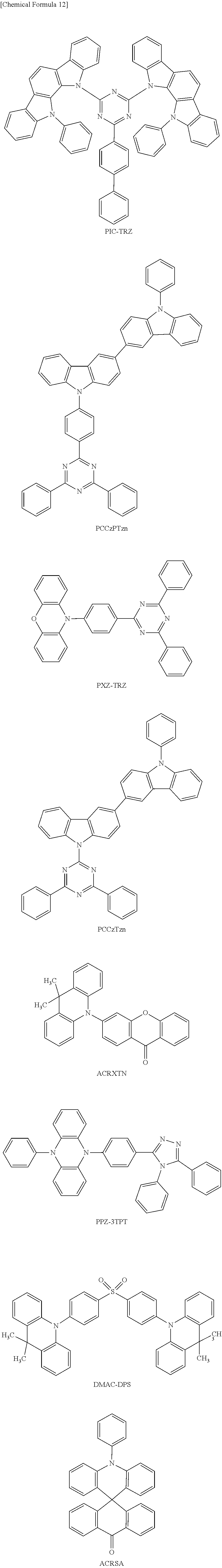

Note that a TADF material is a material having a small difference between the S₁ level and the T₁ level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light emission.

An exciplex whose excited state is formed of two kinds of substances has an extremely small difference between the S₁ level and the T₁ level and functions as a TADF material capable of converting triplet excitation energy into singlet excitation energy.

A phosphorescent spectrum observed at a low temperature (e.g., 77 K to 10 K) is used for an index of the T₁ level. When the level of energy with a wavelength of the line obtained by extrapolating a tangent to the fluorescent spectrum at a tail on the short wavelength side is the S₁ level and the level of energy with a wavelength of the line obtained by extrapolating a tangent to the phosphorescent spectrum at a tail on the short wavelength side is the T₁ level, the difference between the S₁ level and the T₁ level of the TADF material is preferably smaller than or equal to 0.3 eV, further preferably smaller than or equal to 0.2 eV.

When a TADF material is used as the light-emitting substance, the S₁ level of the host material is preferably higher than that of the TADF material. In addition, the T₁ level of the host material is preferably higher than that of the TADF material.

As the host material in the light-emitting layer, various carrier-transport materials such as materials having an electron-transport property and/or materials having a hole-transport property, and the TADF materials can be used. In particular, an organic compound deuterated using one embodiment of the present invention is preferable.

The material having a hole-transport property is preferably an organic compound having an amine skeleton or a π-electron rich heteroaromatic ring skeleton, for example. As the n-electron rich heteroaromatic ring, a condensed aromatic ring having at least one of an acridine skeleton, a phenoxazine skeleton, a phenothiazine skeleton, a furan skeleton, a thiophene skeleton, and a pyrrole skeleton is preferable; specifically, a carbazole ring, a dibenzothiophene ring, or a ring in which an aromatic ring or a heteroaromatic ring is further condensed to a carbazole ring or a dibenzothiophene ring is preferable.

Such an organic compound having a hole-transport property further preferably has any of a carbazole skeleton, a dibenzofuran skeleton, a dibenzothiophene skeleton, and an anthracene skeleton. In particular, an aromatic amine including a substituent having a dibenzofuran ring or a dibenzothiophene ring, an aromatic monoamine having a naphthalene ring, or an aromatic monoamine in which a 9-fluorenyl group is bonded to nitrogen of an amine through an arylene group may be used. Note that the organic compound having a hole-transport property preferably has an N,N-bis(4-biphenyl)amino group to enable manufacturing a light-emitting device with a long lifetime.

Examples of such an organic compound include compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 9,9′-diphenyl-9H,9′H-3,3′-bicarbazole (abbreviation: PCCP); compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. In addition, the organic compounds given as examples of the material having a hole-transport property that can be used for the hole-transport layer can also be used.

As the material having an electron-transport property, for example, a metal complex such as bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); or an organic compound having a π-electron deficient heteroaromatic ring is preferably used. Examples of the organic compound including a π-electron deficient heteroaromatic ring skeleton include an organic compound including a heteroaromatic ring having an azole skeleton, an organic compound including a heteroaromatic ring having a pyridine skeleton, an organic compound including a heteroaromatic ring having a diazine skeleton, and an organic compound including a heteroaromatic ring having a triazine skeleton.

Among the above materials, the organic compound including a heteroaromatic ring having a diazine skeleton (a pyrimidine skeleton, a pyrazine skeleton, or a pyridazine skeleton), the organic compound including a heteroaromatic ring having a pyridine skeleton, and the organic compound including a heteroaromatic ring having a triazine skeleton have high reliability and thus are preferable. In particular, the organic compound including a heteroaromatic ring having a diazine (pyrimidine or pyrazine) skeleton and the organic compound including a heteroaromatic ring having a triazine skeleton have a high electron-transport property to contribute to a reduction in driving voltage. A benzofuropyrimidine skeleton, a benzothienopyrimidine skeleton, a benzofuropyrazine skeleton, and a benzothienopyrazine skeleton are preferable because of their high acceptor property and high reliability.

Examples of the organic compound having a π-electron deficient heteroaromatic ring skeleton include organic compounds having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOS); organic compounds including a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 4,7-diphenyl-1,10-phenthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2-[3-(2-triphenylenyl)phenyl]-1,10-phenanthroline (abbreviation: mTpPPhen), 2-phenyl-9-(2-triphenylenyl)-1,10-phenanthroline (abbreviation: Ph-TpPhen), 2-[4-(9-phenanthrenyl)-1-naphthalenyl]-1,10-phenanthroline (abbreviation: PnNPhen), and 2-[4-(2-triphenylenyl)phenyl]-1,10-phenanthroline (abbreviation: pTpPPhen); organic compounds having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-(3-[3-(N-phenyl-9H-carbazol-3-yl)-9H-carbazol-9-yl]phenyl)dibenzo[f,h]quinoxaline (abbreviation: 2mPCCzPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl) (biphenyl-3-yl)]naphtho[1′,2′:4,5]furo[3,2-d]pyrimidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis(4-[4-(2-naphthyl)phenyl]-6-phenylpyrimidine) (abbreviation: 2,6(NP-PPm)2Py), 6-(biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), and 7-[4-(9-phenyl-9H-carbazol-2-yl)quinazolin-2-yl]-7H-dibenzo[c,g]carbazole (abbreviation: PC-cgDBCzQz); and organic compounds including a heteroaromatic ring having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2-[3-(2,6-dimethyl-3-pyridinyl)-5-(9-phenanthrenyl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn), 2-[4-(2-naphthalenyl)phenyl]-4-phenyl-6-spiro[9H-fluorene-9,9′-[9H]xanthen]-4-yl-1,3,5-triazine (abbreviation: βNP-SFx(4)Tzn), and 2-phenyl-4,6-bis[3-(triphenylsilyl)phenyl]-1,3,5-triazine (abbreviation: mSiTrz). The organic compound including a heteroaromatic ring having a diazine skeleton, the organic compound including a heteroaromatic ring having a pyridine skeleton, and the organic compound including a heteroaromatic ring having a triazine skeleton are preferable because of their high reliability. In particular, the organic compound including a heteroaromatic ring having a diazine (pyrimidine or pyrazine) skeleton and the organic compound including a heteroaromatic ring having a triazine skeleton have a high electron-transport property to contribute to a reduction in driving voltage.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the S₁ level of the fluorescent substance. Therefore, the T₁ level of the TADF material is preferably higher than that of the fluorescent substance.

It is also preferable to use a TADF material emitting light whose wavelength overlaps with the wavelength on the lowest-energy-side absorption band of the fluorescent substance. This is preferable because excitation energy can be smoothly transferred from the TADF material to the fluorescent substance and accordingly light emission can be obtained efficiently.

In order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton that brings about light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transportation or carrier recombination. Here, the luminophore refers to an atomic group (skeleton) that brings about light emission in a fluorescent substance. The luminophore is preferably a skeleton having a π bond, further preferably includes an aromatic ring, and still further preferably includes a condensed aromatic ring or a condensed heteroaromatic ring. Examples of the luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton that is used as the host material, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further condensed to a carbazole skeleton because the HOMO level thereof is higher than that of the host material having a carbazole skeleton by approximately 0.1 eV and thus holes enter the host material easily. In particular, the host material preferably has a dibenzocarbazole skeleton because the HOMO level thereof is higher than that of the host material having a carbazole skeleton by approximately 0.1 eV and thus holes enter the host material easily, the hole-transport property is improved, and the heat resistance is increased. Accordingly, a substance having both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4-(9-phenyl-9H-fluoren-9-yl)biphenyl-4-yl]anthracene (abbreviation: FLPPA), 9-(1-naphthyl)-10-[4-(2-naphthyl)phenyl]anthracene (abbreviation: αN-βNPAnth), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(2-naphthyl)-10-[3-(2-naphthyl)phenyl]anthracene (abbreviation: ON-mpNPAnth), and 1-{4-[10-(biphenyl-4-yl)-9-anthracenyl]phenyl}-2-ethyl-1H-benzimidazole (abbreviation: EtBImPBPhA). In particular, CzPA, cgDBCzPA, 2mBnfPPA, and PCzPA exhibit excellent properties and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property can be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength on the lowest-energy-side absorption band of the light-emitting substance, in which case energy can be transferred smoothly and light emission can be obtained efficiently. The use of such a structure is preferable because the driving voltage can also be reduced.

Note that at least one of the materials forming an exciplex may be a phosphorescent substance. In this case, triplet excitation energy can be efficiently converted into singlet excitation energy by reverse intersystem crossing.

In order to form an exciplex efficiently, a material having an electron-transport property is preferably combined with a material having a hole-transport property and a HOMO level higher than or equal to that of the material having an electron-transport property. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient photoluminescence (PL) lifetime of the mixed film has longer lifetime components or has a larger proportion of delayed components than that of each of the materials, observed by comparison of transient PL of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

The electron-transport layer 114 contains a material having an electron-transport property. The material having an electron-transport property preferably has an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property. An organic compound including a π-electron deficient heteroaromatic ring is preferable as the above organic compound. The organic compound including a π-electron deficient heteroaromatic ring is preferably one or more of an organic compound including a heteroaromatic ring having an azole skeleton, an organic compound including a heteroaromatic ring having a pyridine skeleton, an organic compound including a heteroaromatic ring having a diazine skeleton, and an organic compound including a heteroaromatic ring having a triazine skeleton.

As the organic compounds having an electron-transport property that can be used in the electron-transport layer 114, the organic compound having an electron-transport property in the light-emitting layer 113 and the organic compound mentioned in Embodiment 1 as the organic compound that can be used as the second organic compound in the electron-injection layer 115 can be similarly used. Among the above-described materials, the organic compound including a heteroaromatic ring having a diazine skeleton, the organic compound including a heteroaromatic ring having a pyridine skeleton, and the organic compound including a heteroaromatic ring having a triazine skeleton are especially preferable because of having high reliability. In particular, the organic compound including a heteroaromatic ring having a diazine (pyrimidine or pyrazine) skeleton and the organic compound including a heteroaromatic ring having a triazine skeleton have a high electron-transport property to contribute to a reduction in driving voltage. An organic compound having a phenanthroline skeleton such as mTpPPhen, PnNPhen, or mPPhen2P is preferable, and an organic compound having a phenanthroline dimer structure such as mPPhen2P is further preferable because of its excellent stability. The organic compound having an electron-transport property and high HOMO level, such as 2mPCCzPDBq and DACT-II, is preferable because a light-emitting device with a low driving voltage can be obtained.

The electron-transport layer preferably contains an organic compound having an electron-transport property with an acid dissociation constant pKₐ of less than 4.

The electron-transport layer 114 may have a stacked-layer structure. In the case where the electron-transport layer 114 has a stacked-layer structure, the layer in contact with the light-emitting layer 113 may function as a hole-blocking layer. In the case where the electron-transport layer in contact with the light-emitting layer functions as a hole-blocking layer, the electron-transport layer is preferably formed using a material having a lower HOMO level than a material contained in the light-emitting layer by more than or equal to 0.5 eV.

The electron-injection layer 115 is formed between the electron-transport layer 114 and the second electrode 102. Since the structure of the electron-injection layer 115 has been described in detail in Embodiment 1, the repetitive description thereof is omitted.

The second electrode 102 is an electrode including a cathode. The second electrode 102 may have a stacked-layer structure, in which case a layer in contact with the organic compound layer 103 functions as a cathode. For the cathode, a metal, an alloy, an electrically conductive compound, or a mixture thereof each having a low work function (specifically, lower than or equal to 3.8 eV) or the like can be used. Specific examples of such a cathode material include elements belonging to Group 1 or 2 of the periodic table, such as alkali metals (e.g., lithium (Li) and cesium (Cs)), magnesium (Mg), calcium (Ca), and strontium (Sr), alloys containing these elements (e.g., MgAg and AlLi), compounds containing these elements (e.g., lithium fluoride (LiF), cesium fluoride (CsF), and calcium fluoride (CaF₂)), rare earth metals such as europium (Eu) and ytterbium (Yb), and alloys containing these rare earth metals. However, when the electron-injection layer 115 or a thin film formed using any of the above materials having a low work function is provided between the second electrode 102 and the electron-transport layer, a variety of conductive materials such as Al, Ag, ITO, or indium oxide-tin oxide containing silicon or silicon oxide can be used for the cathode regardless of the work function.

When the second electrode 102 is formed using a material transmitting visible light, the light-emitting device can emit light from the second electrode 102 side. When the first electrode 101 is formed using a material transmitting visible light, the light-emitting device can emit light from the first electrode 101 side.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

Note that in the case of a top-emission light-emitting device, forming a cap layer by evaporation of an organic compound over the second electrode can improve light extraction efficiency. The cap layer may have a single-layer structure or a stacked-layer structure. In the case of a stacked-layer structure, the use of organic compounds with different refractive indexes can further increase the light extraction efficiency.

The organic compound layer 103 can be formed by any of a variety of methods, including a dry process and a wet process. For example, a vacuum evaporation method, a gravure printing method, an offset printing method, a screen printing method, an ink-jet method, a spin coating method, or the like may be used.

Different film formation methods may be used to form the electrodes or the layers described above.

Next, an embodiment of a light-emitting device having a structure where a plurality of light-emitting units are stacked (this type of light-emitting device is also referred to as a stacked or tandem device) is described with reference to FIG. 1B. This light-emitting device includes a plurality of light-emitting units between an anode and a cathode. One light-emitting unit has substantially the same structure as the organic compound layer 103 illustrated in FIG. 1A. In other words, the light-emitting device illustrated in FIG. 1B includes a plurality of light-emitting units, and the light-emitting device illustrated in FIG. 1A includes a single light-emitting unit.

In FIG. 1B, a first light-emitting unit 511 and a second light-emitting unit 512 are stacked between a first electrode 501 and a second electrode 502, and an intermediate layer 513 is provided between the first light-emitting unit 511 and the second light-emitting unit 512. The first electrode 501 and the second electrode 502 correspond, respectively, to the first electrode 101 and the second electrode 102 illustrated in FIG. 1A, and the materials given in the description for FIG. 1A can be used. Furthermore, the first light-emitting unit 511 and the second light-emitting unit 512 may have the same structure or different structures.

The intermediate layer 513 has a function of injecting electrons into one of the light-emitting units and injecting holes into the other of the light-emitting units when voltage is applied between the first electrode 501 and the second electrode 502. That is, in FIG. 1B, the intermediate layer 513 injects electrons into the first light-emitting unit 511 and holes into the second light-emitting unit 512 when voltage is applied such that the potential of the anode becomes higher than the potential of the cathode.

The intermediate layer 513 includes a charge-generation layer. The charge-generation layer includes at least a p-type layer 117. The p-type layer 117 is preferably formed using any of the composite materials given above as examples of materials that can be used for the hole-injection layer 111. The p-type layer 117 maybe formed by stacking a film containing the above-described acceptor material as a material included in the composite material and a film containing a hole-transport material. When a potential is applied to the p-type layer 117, electrons are injected into the electron-transport layer 114 and holes are injected into the cathode; thus, the light-emitting device operates.

Note that the intermediate layer 513 preferably includes one or both of an electron-relay layer 118 and an n-type layer 119 in addition to the p-type layer 117.

The electron-relay layer 118 contains at least a substance having an electron-transport property and has a function of preventing an interaction between the n-type layer 119 and the p-type layer 117 and smoothly transferring electrons. The LUMO level of the substance having an electron-transport property contained in the electron-relay layer 118 is preferably between the LUMO level of an acceptor substance in the p-type layer 117 and the LUMO level of a substance contained in a layer of the electron-transport layer 114 that is in contact with the intermediate layer 513. As a specific value of the energy level, the LUMO level of the substance having an electron-transport property in the electron-relay layer 118 is preferably higher than or equal to −5.0 eV, further preferably higher than or equal to −5.0 eV and lower than or equal to −3.0 eV. Note that as the substance having an electron-transport property in the electron-relay layer 118, a phthalocyanine-based material or a metal complex having a metal-oxygen bond and an aromatic ligand is preferably used.

The n-type layer 119 can be formed using a substance having a high electron-injection property, e.g., an alkali metal, an alkaline earth metal, a rare earth metal, or a compound thereof (an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)).

In the case where the n-type layer 119 contains a substance having an electron-transport property and a donor substance, the donor substance can be an organic compound such as tetrathianaphthacene (abbreviation: TTN), nickelocene, or decamethylnickelocene, as well as an alkali metal, an alkaline earth metal, a rare earth metal, or a compound thereof (e.g., an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)). As the substance having an electron-transport property, a material similar to the above-described material for the electron-transport layer 114 can be used.

Instead of the n-type layer 119, a metal or a metal compound, a first organic compound having a π-electron deficient heteroaromatic ring, and a second organic compound having two or more heteroaromatic rings that are bonded or condensed to each other and include three or more heteroatoms in total, which are described as being used for the electron-injection layer in Embodiment 1, may be formed in the same position as the n-type layer 119. Also in the case of such a structure, a tandem light-emitting device with favorable characteristics can be manufactured.

In the case where the anode-side surface of a light-emitting unit is in contact with the intermediate layer 513, the charge-generation layer of the intermediate layer 513 can also function as a hole-injection layer of the light-emitting unit; therefore, a hole-injection layer is not necessarily provided in the light-emitting unit. In the case where the cathode-side surface of a light-emitting unit is in contact with the intermediate layer 513, the intermediate layer 513 can also function as an electron-injection layer of the light-emitting unit; therefore, an electron-injection layer is not necessarily provided in the light-emitting unit.

The light-emitting device having two light-emitting units is described with reference to FIG. 1B; however, one embodiment of the present invention can also be applied to a light-emitting device in which three or more light-emitting units are stacked. With a plurality of light-emitting units partitioned by the intermediate layer 513 between a pair of electrodes as in the light-emitting device of this embodiment, it is possible to provide a long-life element that can emit light with high luminance at a low current density. A light-emitting apparatus that can be driven at a low voltage and has low power consumption can also be provided.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as the whole.

The organic compound layer 103, the first light-emitting unit 511, the second light-emitting unit 512, the layers such as the intermediate layer 513, and the electrodes that are described above can be formed by a method such as an evaporation method (including a vacuum evaporation method), a droplet discharge method (also referred to as an ink-jet method), a coating method, or a gravure printing method. A low molecular material, a middle molecular material (including an oligomer and a dendrimer), or a high molecular material may be included in the above components.

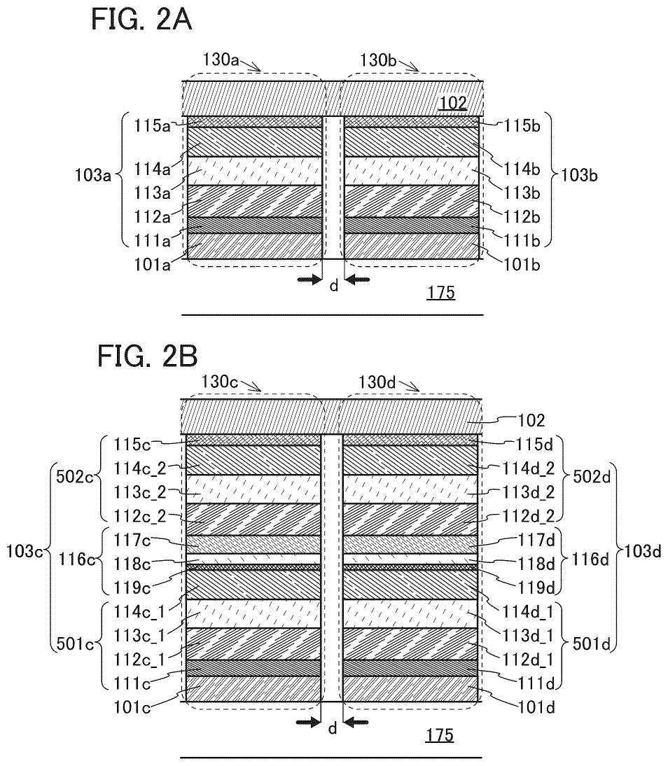

FIG. 2A illustrates two adjacent light-emitting devices (a light-emitting device 130a and a light-emitting device 130b) included in a display apparatus of one embodiment of the present invention.

The light-emitting device 130a includes an organic compound layer 103a between a first electrode 101a over an insulating layer 175 and the second electrode 102 facing the first electrode 101a. The organic compound layer 103a includes a hole-injection layer 111a, a hole-transport layer 112a, a light-emitting layer 113a, an electron-transport layer 114a, and an electron-injection layer 115a, but may have a different stacked-layer structure.

The light-emitting device 130b includes an organic compound layer 103b between a first electrode 101b over the insulating layer 175 and the second electrode 102 facing the first electrode 101b. The organic compound layer 103b includes a hole-injection layer 111b, a hole-transport layer 112b, a light-emitting layer 113b, an electron-transport layer 114b, and an electron-injection layer 115b, but may have a different stacked-layer structure.

The structures of the electron-transport layer 114a and the electron-injection layer 115a in the light-emitting device 130a and the structures of the electron-transport layer 114b and the electron-injection layer 115b in the light-emitting device 130b are preferably those described in Embodiment 1.

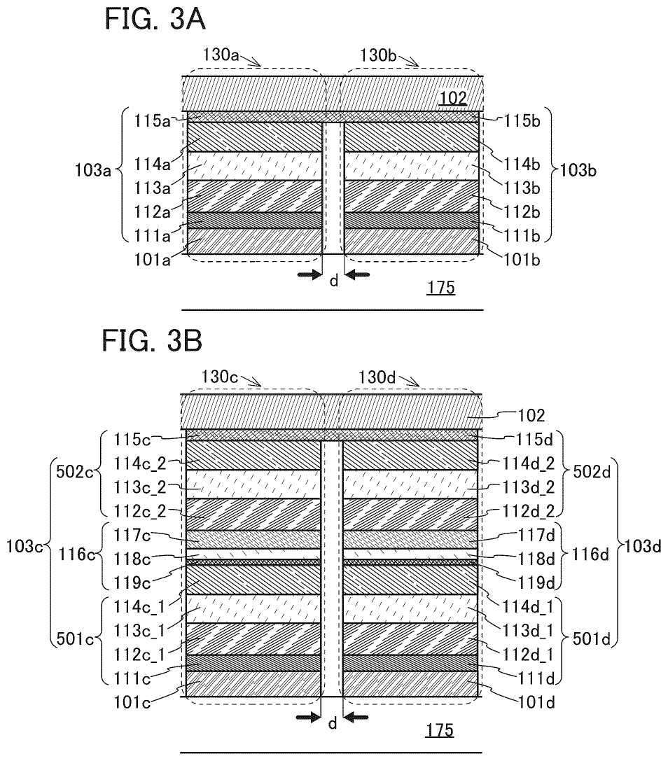

The second electrode 102 is preferably one layer shared by the light-emitting devices 130a and 130b. The organic compound layers 103a and 103b are independent of each other because processing by a photolithography method is performed after the electron-injection layer 115a is formed and after the electron-injection layer 115b is formed. In the light-emitting device of one embodiment of the present invention, even though processing by a photolithography method is performed after the electron-injection layer 115a is formed and after the electron-injection layer 115b is formed, the light-emitting device can have favorable characteristics. Note that as illustrated in FIG. 3A, the electron-injection layers 115a and 115b may be one layer shared by the light-emitting devices 130a and 130b.

The end portions (outlines) of the layers in the organic compound layer 103a are substantially aligned in the direction perpendicular to the substrate due to the processing by a photolithography method. Furthermore, the end portions (outlines) of the layers in the organic compound layer 103b are substantially aligned in the direction perpendicular to the substrate due to the processing by a photolithography method.

There is a space d between the organic compound layer 103a and the organic compound layer 103b due to the processing by a photolithography method. Since the organic compound layers are processed by a photolithography method, the distance between the first electrode 101a and the first electrode 101b can be small as compared with the case where mask vapor deposition is performed, and can be greater than or equal to 0.5 pm and less than or equal to 5 pm.

FIG. 2B illustrates two adjacent tandem light-emitting devices (a light-emitting device 130c and a light-emitting device 130d) manufactured by a photolithography method.

The light-emitting device 130c includes an organic compound layer 103c between a first electrode 101c over the insulating layer 175 and the second electrode 102. The organic compound layer 103c has a structure where a first light-emitting unit 501c and a second light-emitting unit 502c are stacked with an intermediate layer 116c therebetween. Although FIG. 2B illustrates an example where the two light-emitting units are stacked, three or more light-emitting units may be stacked. The first light-emitting unit 501c includes a hole-injection layer 111c, a first hole-transport layer 112c_1, a first light-emitting layer 113c_1, and a first electron-transport layer 114c_1. The intermediate layer 116c includes a p-type layer 117c, an electron-relay layer 118c, and an n-type layer 119c. The electron-relay layer 118c is not necessarily provided. The second light-emitting unit 502c includes a second hole-transport layer 112c_2, a second light-emitting layer 113c_2, a second electron-transport layer 114c_2, and an electron-injection layer 115c.

The light-emitting device 130d includes an organic compound layer 103d between a first electrode 101d over the insulating layer 175 and the second electrode 102. The organic compound layer 103d has a structure where a first light-emitting unit 501d and a second light-emitting unit 502d are stacked with an intermediate layer 116d therebetween. Although FIG. 2B illustrates an example where the two light-emitting units are stacked, three or more light-emitting units may be stacked. The first light-emitting unit 501d includes a hole-injection layer 111d, a first hole-transport layer 112d_1, a first light-emitting layer 113d_1, and a first electron-transport layer 114d_1. The intermediate layer 116d includes a p-type layer 117d, an electron-relay layer 118d, and an n-type layer 119d. The electron-relay layer 118d is not necessarily provided. The second light-emitting unit 502d includes a second hole-transport layer 112d_2, a second light-emitting layer 113d_2, a second electron-transport layer 114d_2, and an electron-injection layer 115d.

In the light-emitting devices 130c and 130d, the electron-injection layers 115c and 115d preferably have the structure described in Embodiment 1.

Note that the second electrode 102 is preferably one layer shared by the light-emitting devices 130c and 130d. The organic compound layers 103c and 103d are independent of each other because processing by a photolithography method is performed after the electron-injection layer 115c is formed and after the electron-injection layer 115d is formed. In the light-emitting device of one embodiment of the present invention, even though processing by a photolithography method is performed after the electron-injection layer 115c is formed and after the electron-injection layer 115d is formed, the light-emitting device can have favorable characteristics. Note that as illustrated in FIG. 3B, the electron-injection layers 115c and 115d may be one layer shared by the light-emitting devices 130c and 130d.

The end portions (outlines) of the layers in the organic compound layer 103c are substantially aligned in the direction perpendicular to the substrate due to the processing by a photolithography method. Furthermore, the end portions (outlines) of the layers in the organic compound layer 103d are substantially aligned in the direction perpendicular to the substrate due to the processing by a photolithography method.

There is the space d between the organic compound layer 103c and the organic compound layer 103d due to the processing by a photolithography method. Since the organic compound layers are processed by a photolithography method, the distance between the first electrode 101c and the first electrode 101d can be small as compared with the case where mask vapor deposition is performed, and can be greater than or equal to 0.5 pm and less than or equal to 5 pm.

In the light-emitting device of one embodiment of the present invention, since the organic compound layer is processed by a photolithography method, the organic compound layer can be processed with a sufficient accuracy to manufacture a high-resolution display apparatus. Furthermore, since a lithography process can be performed on the electron-injection layer far from the light-emitting layer without contamination by an alkali metal, the light-emitting device can have favorable characteristics. As described above, the light-emitting device of one embodiment of the present invention having the above-described structure enables a high-resolution display apparatus and can have favorable characteristics.

Since the organic compound layer in the light-emitting device of one embodiment of the present invention is processed at once by a photolithography method, all the layers included in the organic compound layer have substantially the same outline. Here, “substantially the same” in this specification means, supposing that the organic compound layer includes a layer A and a layer B, a difference between an outline A of the layer A and an outline B of the layer B is within 5% of the width of the organic compound layer along a line orthogonal to the compared portions of the outlines. In the case where an end surface of the organic compound layer has a tapered shape, a continuous change of the outline is allowed.

The structure of this embodiment can be used in combination with any of the other structures as appropriate.

### Embodiment 3

In this embodiment, display apparatuses of one embodiment of the present invention will be described.

The display apparatus in this embodiment can be a high-resolution display apparatus. Thus, the display apparatus in this embodiment can be used for display portions of information terminals (wearable devices) such as watch-type and bracelet-type information terminals and display portions of wearable devices that can be worn on a head, such as a VR device like a head mounted display (HMD) and a glasses-type AR device.

The display apparatus in this embodiment can be a high-definition display apparatus or a large-sized display apparatus. Accordingly, the display apparatus in this embodiment can be used for display portions of a digital camera, a digital video camera, a digital photo frame, a mobile phone, a portable game console, a portable information terminal, and an audio reproducing device, in addition to display portions of electronic devices with a relatively large screen, such as a television device, desktop and notebook personal computers, a monitor of a computer and the like, digital signage, and a large game machine such as a pachinko machine.

### [Display Module]

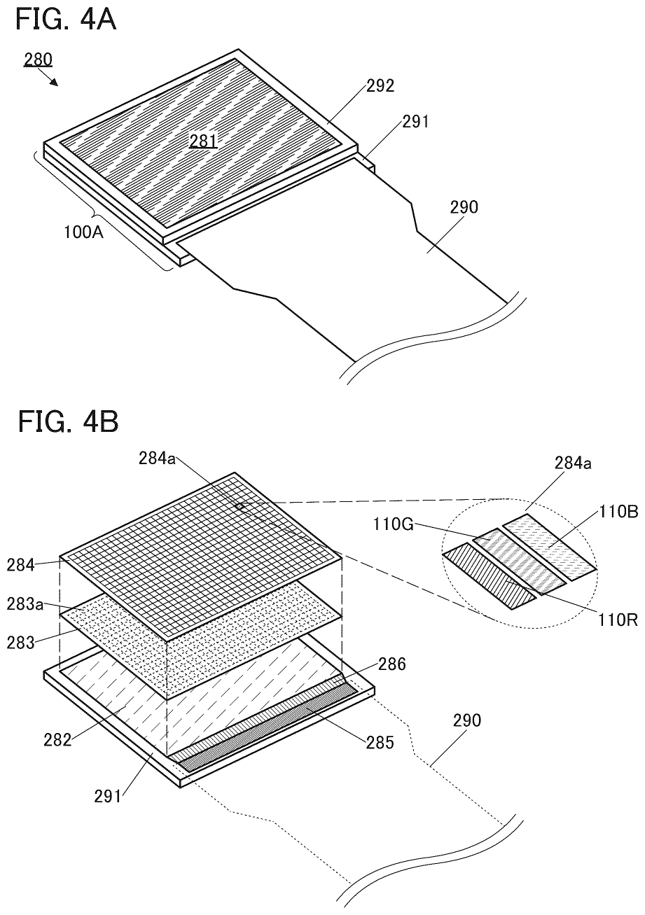

FIG. 4A is a perspective view of a display module 280. The display module 280 includes a display apparatus 100A and an FPC 290. Note that the display apparatus included in the display module 280 is not limited to the display apparatus 100A and may be any of display apparatuses 100B to 100E2 described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 4B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion over the substrate 291 not overlapping with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 4B. The pixels 284a can employ any of the structures described in the above embodiments.

The pixel circuit portion 283 includes a plurality of pixel circuits 283a arranged periodically.

One pixel circuit 283a is a circuit that controls driving of a plurality of elements included in one pixel 284a.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure where one or both of the pixel circuit portion 283 and the circuit portion 282 are stacked below the pixel portion 284; hence, the aperture ratio (effective display area ratio) of the display portion 281 can be significantly high.

Such a display module 280 has extremely high resolution, and thus can be suitably used for a VR device such as an HMD or a glasses-type AR device. For example, even in the case of a structure where the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic devices including a relatively small display portion.

### [Display Apparatus 100A]

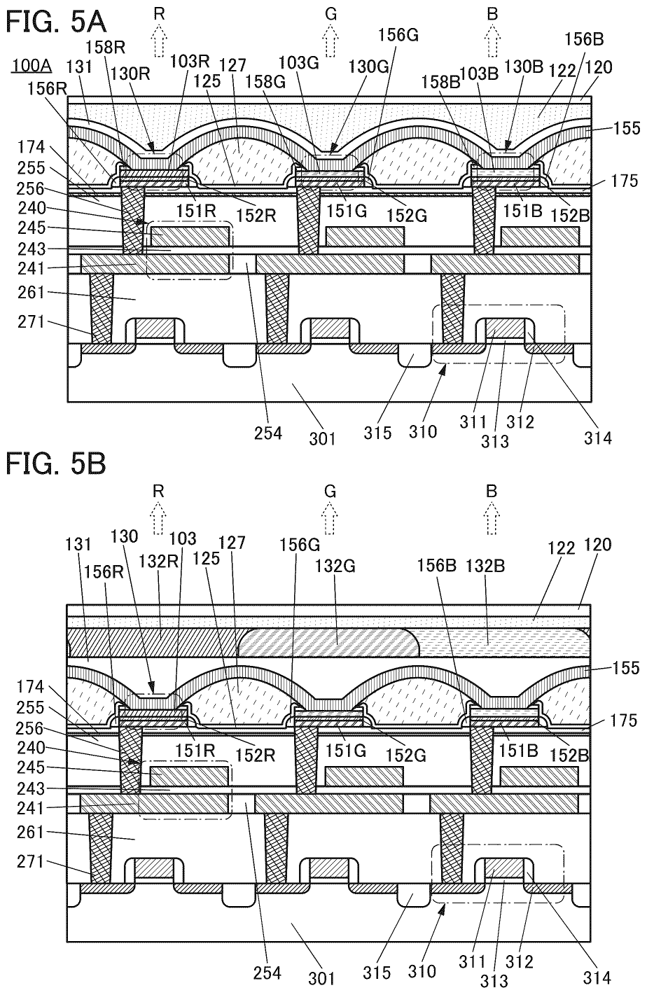

The display apparatus 100A illustrated in FIG. 5A includes a substrate 301, light-emitting devices 130R, 130G, and 130B, a capacitor 240, and a transistor 310.

The substrate 301 corresponds to the substrate 291 in FIGS. 4A and 4B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. An insulating layer 174 is provided over the insulating layer 255. The insulating layer 175 is provided over the insulating layer 174. The light-emitting devices 130R, 130G, and 130B are provided over the insulating layer 175. An insulator is provided in regions between adjacent light-emitting devices.

An insulating layer 156 (insulating layers 156R, 156G, and 156B) is provided to include regions overlapping with the side surfaces of a conductive layer 151 (conductive layers 151R, 151G, and 151B) and a conductive layer 152 (conductive layers 152R, 152G, and 152B). A sacrificial layer 158 (sacrificial layers 158R, 158B, and 158G) is positioned over the organic compound layer 103 (the organic compound layers 103R, 103G, and 103B). An inorganic insulating layer 125 and an insulating layer 127 over the inorganic insulating layer 125 are preferably provided between the adjacent light-emitting devices 130.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. Any of a variety of conductive materials can be used for the plugs.

A protective layer 131 is provided over the light-emitting devices 130R, 130G, and 130B. A substrate 120 is attached to the protective layer 131 with a resin layer 122. Embodiment 2 can be referred to for the details of the light-emitting device 130 and the components thereover up to the substrate 120. The substrate 120 corresponds to the substrate 292 in FIG. 4A.

FIG. 5B illustrates a variation example of the display apparatus 100A illustrated in FIG. 5A. The display apparatus illustrated in FIG. 5B includes a coloring layer 132R, a coloring layer 132G, and a coloring layer 132B, and each of the light-emitting devices 130 includes a region overlapping with one of the coloring layers 132R, 132G, and 132B. In the display apparatus illustrated in FIG. 5B, the light-emitting device 130 can emit white light, for example. The coloring layer 132R, the coloring layer 132G, and the coloring layer 132B can transmit red light, green light, and blue light, respectively, for example.

### [Display Apparatus 100B]

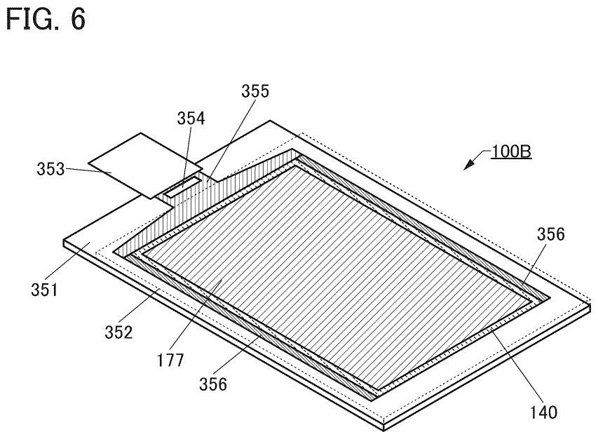

FIG. 6 is a perspective view of the display apparatus 100B.

In the display apparatus 100B, a substrate 352 and a substrate 351 are attached to each other. In FIG. 6, the substrate 352 is denoted by a dashed line.

The display apparatus 100B includes a pixel portion 177, a connection portion 140, a circuit 356, a wiring 355, and the like. FIG. 6 illustrates an example where an IC 354 and an FPC 353 are mounted on the display apparatus 100B. Thus, the structure illustrated in FIG. 6 can be regarded as a display module including the display apparatus 100B, the integrated circuit (IC), and the FPC. Here, a display apparatus in which a substrate is equipped with a connector such as an FPC or mounted with an IC is referred to as a display module.

The connection portion 140 is provided outside the pixel portion 177. The number of connection portions 140 may be one or more. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 6 illustrates an example where the IC 354 is provided over the substrate 351 by a chip on glass (COG) method, a chip on film (COF) method, or the like. An IC including a scan line driver circuit, a signal line driver circuit, or the like can be used as the IC 354, for example. Note that the display apparatus 100B and the display module are not necessarily provided with an IC. Alternatively, the IC may be mounted on the FPC by a COF method, for example.

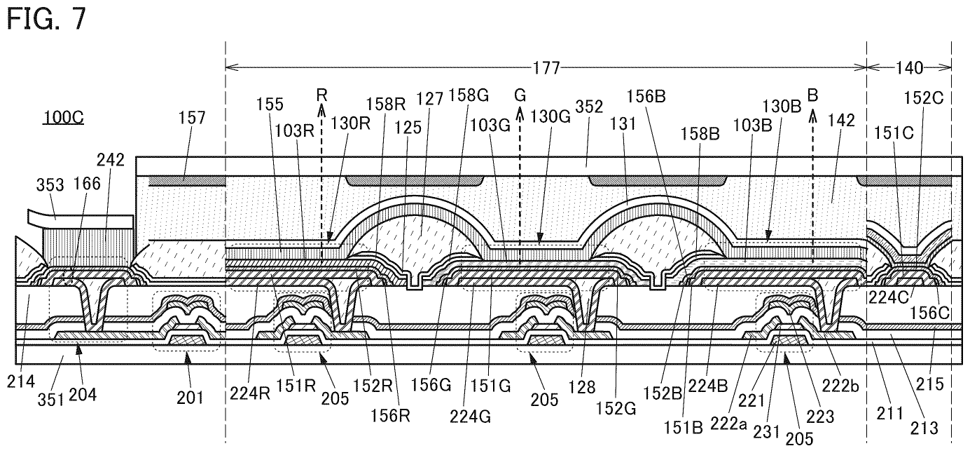

FIG. 7 illustrates the display apparatus 100C as an example of cross sections of part of a region including the FPC 353, part of the circuit 356, part of the pixel portion 177, part of the connection portion 140, and part of a region including an end portion of the display apparatus 100B in FIG. 6.

### [Display Apparatus 100C]

The display apparatus 100C illustrated in FIG. 7 includes a transistor 201, a transistor 205, the light-emitting device 130R emitting red light, the light-emitting device 130G emitting green light, the light-emitting device 130B emitting blue light, and the like between the substrate 351 and the substrate 352.

Embodiment 2 can be referred to for the details of the light-emitting devices 130R, 130G, and 130B.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B.

The conductive layer 224R is connected to a conductive layer 222b included in the transistor 205 through an opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G, and the insulating layer 156G in the light-emitting device 130G are not described in detail because they are respectively similar to the conductive layers 224R, 151R, and 152R, and the insulating layer 156R in the light-emitting device 130R; the same applies to the conductive layers 224B, 151B, and 152B, and the insulating layer 156B in the light-emitting device 130B.

The conductive layers 224R, 224G, and 224B each have a depressed portion covering the opening provided in the insulating layer 214. A layer 128 is embedded in the depressed portion.

The layer 128 has a function of filling the depressed portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depressed portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material that can be used for the insulating layer 127, for example.

The protective layer 131 is provided over the light-emitting devices 130R, 130G, and 130B. The protective layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 7, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided not to overlap with the light-emitting device. Alternatively, the space may be filled with a resin other than the frame-like adhesive layer 142.

FIG. 7 illustrates an example where the connection portion 140 includes a conductive layer 224C obtained by processing the same conductive film as the conductive layers 224R, 224G, and 224B; a conductive layer 151C obtained by processing the same conductive film as the conductive layers 151R, 151G, and 151B; and a conductive layer 152C obtained by processing the same conductive film as the conductive layers 152R, 152G, and 152B. In the example illustrated in FIG. 7, an insulating layer 156C is provided to include a region overlapping with the side surface of the conductive layer 151C.

The display apparatus 100C has a top-emission structure. Light from the light-emitting device is emitted toward the substrate 352. For the substrate 352, a material having a high visible-light-transmitting property is preferably used. A pixel electrode contains a material reflecting visible light, and a counter electrode (a common electrode 155) contains a material transmitting visible light.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

An inorganic insulating film is preferably used as each of the insulating layers 211, 213, and 215.

An organic insulating layer is suitable as the insulating layer 214 functioning as a planarization layer.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate.

A connection portion 204 is provided in a region of the substrate 351 not overlapping with the substrate 352. In the connection portion 204, one of the source electrode and the drain electrode of the transistor 201 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Apparatus 100D]

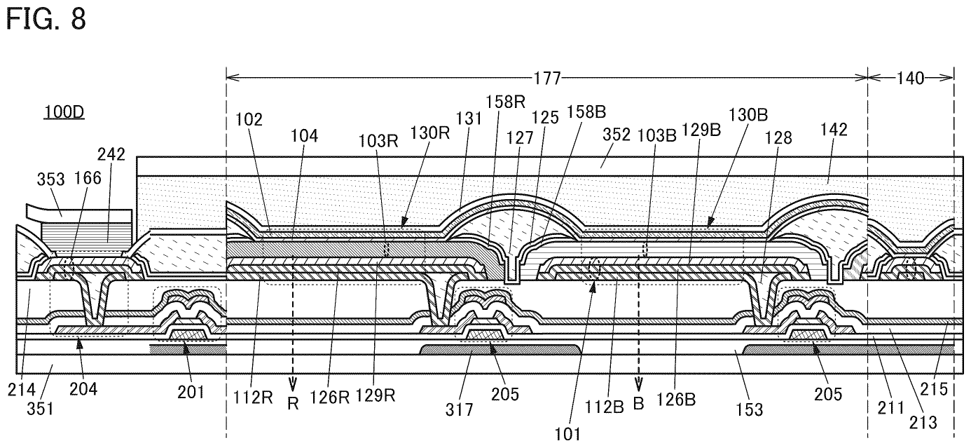

The display apparatus 100D illustrated in FIG. 8 differs from the display apparatus 100C illustrated in FIG. 7 mainly in having a bottom-emission structure.

Light from the light-emitting device is emitted toward the substrate 351. For the substrate 351, a material having a high visible-light-transmitting property is preferably used. By contrast, there is no limitation on the light-transmitting property of a material used for the substrate 352.

A light-blocking layer 317 is preferably formed between the substrate 351 and the transistor 201 and between the substrate 351 and the transistor 205. FIG. 8 illustrates an example where the light-blocking layer 317 is provided over the substrate 351, an insulating layer 153 is provided over the light-blocking layer 317, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material having a high visible-light-transmitting property is used for each of the conductive layers 112R, 112B, 126R, 126B, 129R, and 129B. A material reflecting visible light is preferably used for the second electrode 102.

Although not illustrated in FIG. 8, the light-emitting device 130G is also provided.

Although FIG. 8 and the like illustrate an example where the top surface of the layer 128 includes a flat portion, the shape of the layer 128 is not particularly limited.

### [Display Apparatus 100D2]

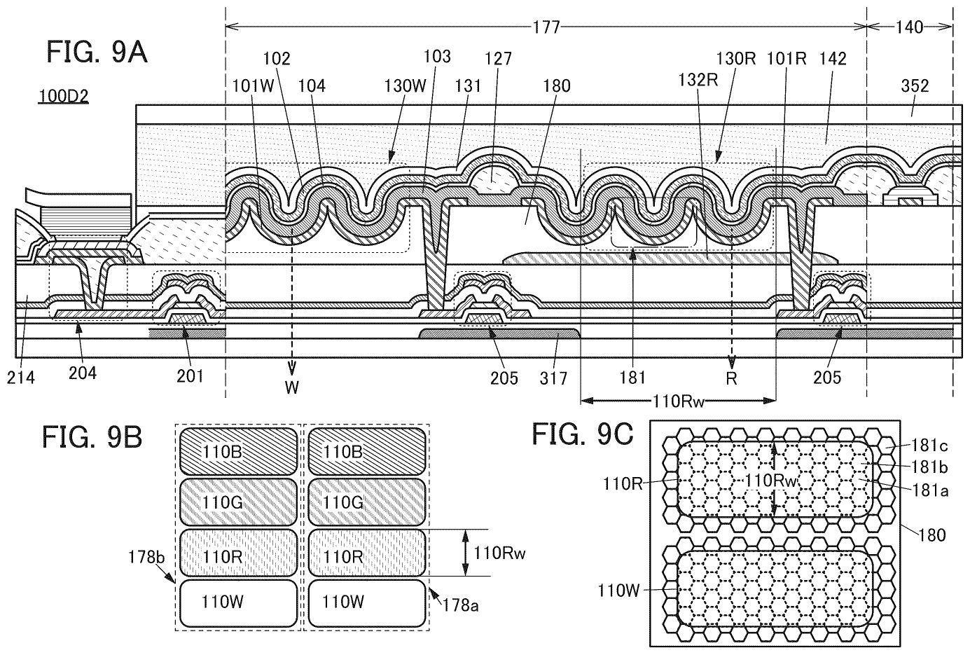

The display apparatus 100D2 illustrated in FIG. 9A is an example of a bottom-emission display apparatus different from the display apparatus 100D illustrated in FIG. 8. The display apparatus 100D2 differs from the display apparatus 100D in including an organic resin layer 180. The display apparatus 100D2 illustrated in FIG. 9A includes the substrate 301, the light-emitting device 130R, and a light-emitting device 130W. Note that the reference numerals of the components that are the same as those in FIG. 8 are sometimes omitted and the description for FIG. 8 is referred to for the details of such components.

FIG. 9B is a top-view layout of a pixel 178 (pixels 178a and 178b) including subpixels 110 (subpixels 110R, 110G, 110B and 110W), and FIG. 9C is a top view of the organic resin layer 180 in a region where the subpixels 110R and 110G included in the pixel 178 are formed. The width between the light-blocking layers 317 corresponds to a width 110Rw in a light-emitting region of the subpixel 110R.

As illustrated in FIG. 9A, the organic resin layer 180 is provided over the insulating layer 214. As illustrated in FIG. 9C and the region surrounded by the dashed-dotted line in FIG. 9A, the organic resin layer 180 includes a depressed portion 181 (depressed portions 181a and 181b) having a curved surface at least in a region where the subpixel is formed. Note that the depressed portion 181 may be provided outside the light-emitting region, like a depressed portion 181c. With the depressed portion 181c, light emission caused in a region overlapping with the light-blocking layer 317 or light travelled into the region overlapping with the light-blocking layer 317 can be refracted and extracted from the light-emitting region, whereby emission efficiency can be improved.

A plurality of depressed portions 181 may be formed in a matrix. The depressed portions 181a and 181b may be provided in contact with each other or may be provided to have a flat surface therebetween.

Although the top surface shape and the cross-sectional shape of the depressed portion are hexagonal (FIG. 9C) and semicircular (FIG. 9A), respectively, other shapes may be employed as needed. Examples of the top surface shape of the depressed portion include polygons such as a triangle, a tetragon (including a rectangle and a square), and a pentagon; polygons with rounded corners; an ellipse; and a circle.

An insulating layer containing an organic material can be used as the organic resin layer 180. Examples of material used for the organic resin layer 180 include an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. The organic resin layer 180 may be formed using an organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin.

A photosensitive resin can also be used for the organic resin layer 180. A photoresist may be used for the photosensitive resin. As the photosensitive resin, a positive photosensitive material or a negative photosensitive material can be used.

The organic resin layer 180 may contain a material absorbing visible light. For example, the organic resin layer 180 itself may be made of a material absorbing visible light, or the organic resin layer 180 may contain a pigment absorbing visible light. For example, the organic resin layer 180 can be formed using a resin that can be used as a color filter transmitting red, blue, or green light and absorbing light of the other colors; or a resin that contains carbon black as a pigment and functions as a black matrix.

The first electrode 101 (first electrodes 101R and 101W) is over the organic resin layer 180 and the organic compound layer 103 is over the first electrode 101. End portions of the first electrode 101 and the organic compound layer 103 may be covered with the insulating layer 127.

The first electrode 101 formed over the organic resin layer 180 also has a depressed portion along the depressed portion of the organic resin layer 180. The organic compound layer 103 formed over the first electrode 101 also has a depressed portion along the depressed portion of the first electrode 101. A common layer 104 formed over the organic compound layer 103 also has a depressed portion along the depressed portion of the organic compound layer 103. The second electrode 102 formed over the common layer 104 also has a depressed portion along the depressed portion of the common layer 104. That is, the depressed portions of the organic resin layer 180, the first electrode 101, the organic compound layer 103, the common layer 104, and the second electrode 102 overlap with each other.

The common layer 104 is over the organic compound layer 103 and the insulating layer 127, and the second electrode 102 is over the common layer 104. The protective layer 131 is provided over the second electrode 102 and attached to the substrate 352 with the adhesive layer 142.

Although the light-emitting devices 130G and 130B are not illustrated in FIGS. 9A to 9C, the light-emitting devices 130G and 130B are also provided.

The light-emitting apparatus of one embodiment of the present invention including the above-described organic resin layer 180 includes the organic compound containing deuterium in the organic compound layer 103 as described in Embodiment 1, whereby an organic semiconductor device with high emission efficiency, high reliability, a low driving voltage, and low power consumption can be provided owing to an indivisible effect of the organic resin layer 180 and the organic compound of the present application.

### [Display Apparatus 100E]

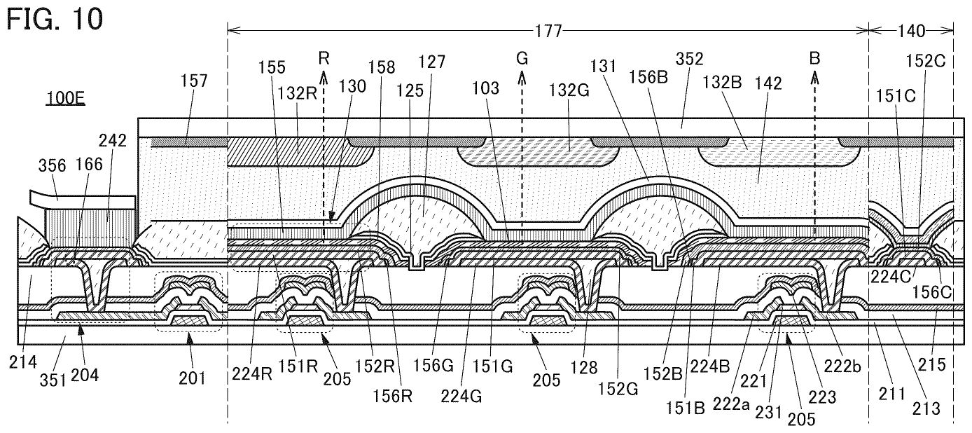

The display apparatus 100E illustrated in FIG. 10 is a variation example of the display apparatus 100C illustrated in FIG. 7 and differs from the display apparatus 100C mainly in including the coloring layers 132R, 132G, and 132B.

In the display apparatus 100E, the light-emitting device 130 includes a region overlapping with one of the coloring layers 132R, 132G, and 132B. The coloring layers 132R, 132G, and 132B can be provided on a surface of the substrate 352 on the substrate 351 side. End portions of the coloring layers 132R, 132G, and 132B can overlap with the light-blocking layer 157.

In the display apparatus 100E, the light-emitting device 130 can emit white light, for example. The coloring layer 132R, the coloring layer 132G, and the coloring layer 132B can transmit red light, green light, and blue light, respectively, for example. Note that in the display apparatus 100E, the coloring layers 132R, 132G, and 132B may be provided between the protective layer 131 and the adhesive layer 142.

### [Display Apparatus 100E2]

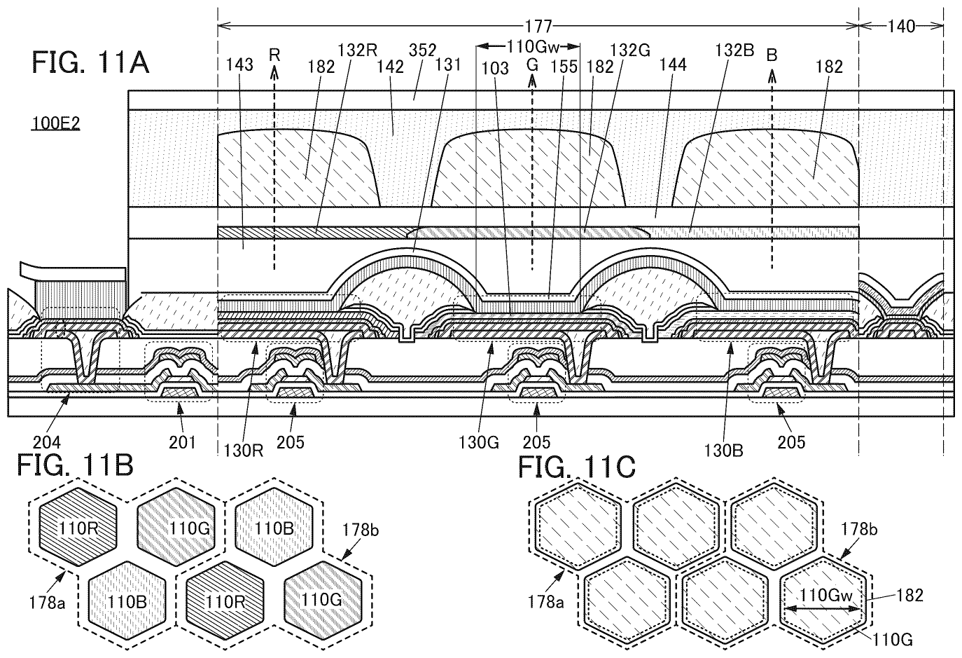

The display apparatus 100E2 illustrated in FIG. 11A is a variation example of the display apparatus 100E illustrated in FIG. 10 and includes microlenses 182 over the coloring layers 132R, 132G, and 132B. Note that the reference numerals of the components that are the same as those in FIG. 10 are sometimes omitted and the description for FIG. 10 is referred to for the details of such components.

FIG. 11B is a top-view layout of the pixel 178 (the pixels 178a and 178b) including the subpixel 110 (the subpixels 110R, 110G, and 110B), and FIG. 11C is a top view of the microlens 182 in a region where the subpixels 110R and 110G included in the pixel 178 are formed. Note that the width of a region where the common electrode 155 and the organic compound layer 103 are in contact with each other corresponds to a width 110Gw in a light-emitting region of the subpixel 110G.

In the display apparatus 100E2 illustrated in FIG. 9A, a planarization film 143 is provided over the protective layer 131, and the coloring layers 132R, 132G, and 132B are provided over the planarization film 143. A planarization film 144 is provided to cover the coloring layers 132R, 132G, and 132B. The microlenses 182 are provided over the planarization film 144.

Note that as illustrated in FIG. 11C, the microlens 182 is preferably provided for each of the subpixels in a region where the subpixel is formed.

Although the top surface shape of the microlens 182 is illustrated as a hexagon in FIG. 11C, other shapes may be employed as needed. Examples of the top surface shape of the depressed portion include polygons such as a triangle, a tetragon (including a rectangle and a square), and a pentagon; polygons with rounded corners; an ellipse; and a circle.

The microlens 182 can be formed using a material similar to that for the organic resin layer 180.

The light-emitting apparatus of one embodiment of the present invention including the above-described microlens 182 includes the organic compound containing deuterium in the organic compound layer 103 as described in Embodiment 1, whereby an organic semiconductor device with high emission efficiency, high reliability, a low driving voltage, and low power consumption, which is suitable for a mobile display, can be provided owing to an indivisible effect of the microlens 182 and the organic compound of the present application.

This embodiment can be combined as appropriate with any of the other embodiments or examples. In this specification, in the case where a plurality of structure examples are shown in one embodiment, the structure examples can be combined as appropriate.

### Example 11

### Synthesis Example 1

In this example, a method for synthesizing 8-chloro-4-hydroxy[1]benzofuro[3,2-d]pyrimidine-2-d₁, which is the organic compound of one embodiment of the present invention represented by Structural Formula (100) in Embodiment 1, will be described. Note that the organic compound represented by Structural Formula (100) and 8-chloro[1]benzofuro[3,2-d]pyrimidine-4(1H)one-2-di represented by Structural Formula (100-1) exist as a mixture in an equilibrium state represented by Structural Formula (100-2).

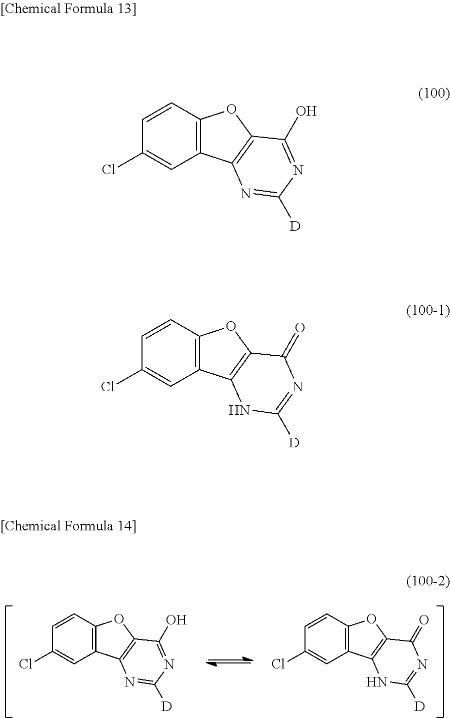

### Step 1: Synthesis of 8-chloro-4-hydroxy[1]benzofuro[3,2-d]pyrimidine-2-d₁

Into an autoclave container were put 9.3 g (39 mmol) of 4,8-dichloro[1]benzofuro[3,2-d]pyrimidine, 31 mL of 2-propanol, 310 mL of heavy water, and 7.59 g (3.9 mmol) of 10% Pt/C. A lid was put on the autoclave container and the air in the container was replaced with Ar. The autoclave container was set on a mounting rack, a cylindrical heater was attached to the container, and the temperature of the heater (external temperature) was raised to 120° C. while the mixture was stirred. After the temperature reached 120° C., heating was performed for 43 hours. After the 43-hour heating, the mixture was cooled down to room temperature and left overnight. Then, the supernatant fluid of this mixed solution was removed by decantation. DMF was added to the remaining solid. Stirring was performed for approximately 1 hour to dissolve part of the solid. The mixed solution in which the solid was dissolved was filtered with Celite to obtain a filtrate. After the filtration, DMF was further added to the Celite to dissolve a target substance from a solid that was separated over the Celite by the filtration, so that a cleaning solution was obtained. The filtrate and the cleaning solution were mixed to give approximately 500 mL of a solution. Approximately 3 L of water was added to this solution, so that a suspension was obtained. This suspension was filtered to give a reddish orange solid. The obtained solid was dried at approximately 80° C. under reduced pressure. After the drying, 4.0 g of a target substance was obtained in a yield of 46%. The synthesis scheme of 8-chloro-4-hydroxy[1]benzofuro[3,2-d]pyrimidine-2-di (or 8-chloro[1]benzofuro[3,2-d]pyrimidine-4(1H)one-2-di) obtained in Step 1 is shown in Formula (a-1) below.

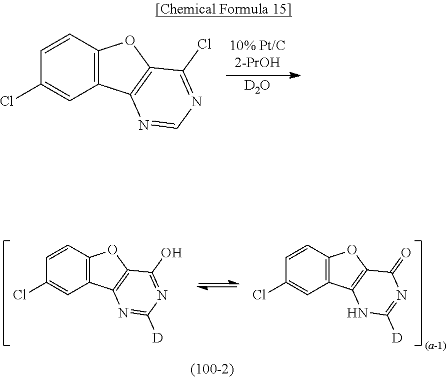

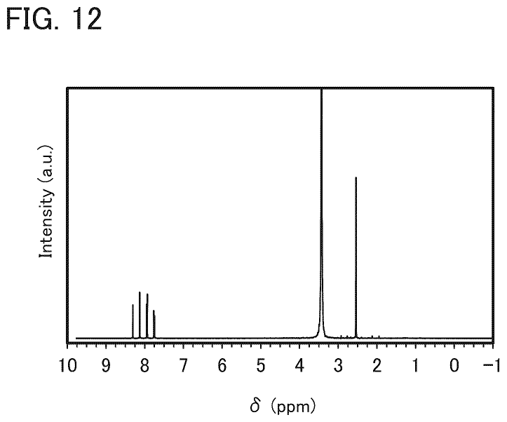

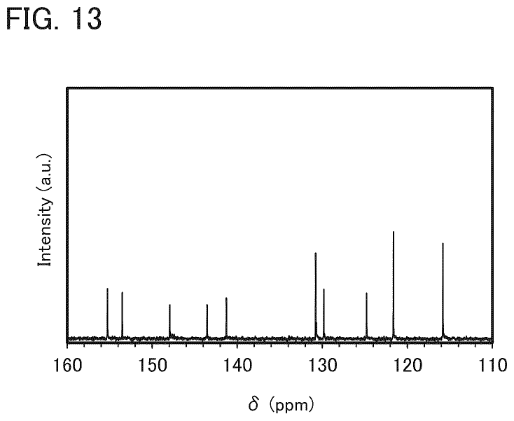

Results of analysis by nuclear magnetic resonance spectroscopy (¹H-NMR and ¹³C-NMR) of the reddish orange solid obtained in Step 1 are shown below. FIG. 12 shows a ¹H-NMR chart, and FIG. 13 shows a ¹³C-NMR chart. The results show that 8-chloro-4-hydroxy[1]benzofuro[3,2-d]pyrimidine-2-d₁ (or 8-chloro[1]benzofuro[3,2-d]pyrimidine-4(1H)one-2-d₁) was synthesized.

¹H-NMR. δ (DIMSO-d₆): 8.31 (s, 0.37H, deuteration rate: 63%), 8.13 (d, 1H, J=7.5 Hz), 7.93-7.95 (m, 1H), 7.74-7.77 (m, 1H).

¹³C-NMR. δ (DIMSO-d₆): 155.3, 153.5, 147.9, 143.5, 141.3, 130.8, 130.7, 129.8, 124.8, 121.6, 115.8.

Next, 8-chloro-4-hydroxy[1]benzofuro[3,2-d]pyrimidine-2-d₁ (or 8-chloro[1]benzofuro[3,2-d]pyrimidine-4(1H)one-2-di) obtained in this example was analyzed by liquid chromatography mass spectrometry (abbreviation: LC/MS).

In the LC/MS analysis, liquid chromatography (LC) separation was performed with ACQUITY UPLC H-Class Plus manufactured by Waters Corporation, and mass spectrometry (MS) analysis was performed with SELECT SERIES Cyclic IMS manufactured by Waters Corporation.

In the LC separation, a given column was used at a column temperature of 40° C., an appropriate solvent was selected for solution sending, a sample was prepared by dissolving 8-chloro-4-hydroxy[1]benzofuro[3,2-d]pyrimidine-2-d₁ (or 8-chloro[1]benzofuro[3,2-d]pyrimidine-4(1H)one-2-d₁) in an organic solvent at a given concentration, and the injection amount of the sample was 5.0 μL.

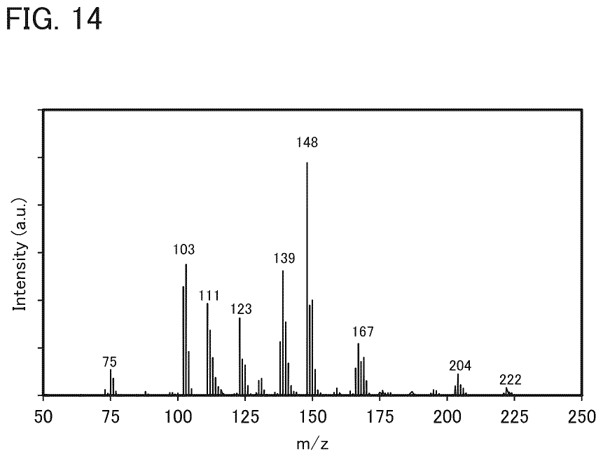

Note that MS² measurement of m/z=221.01 corresponding to the exact mass of 8-chloro-4-hydroxy[1]benzofuro[3,2-d]pyrimidine-2-di (or 8-chloro[1]benzofuro[3,2-d]pyrimidine-4(1H)one-2-di) was performed by a PRM method. For setting of the PRM, the mass range of a target ion was set to m/z=221.01±2.0 (isolation window=4) and detection was performed in a positive mode. The measurement was performed with energy (normalized collision energy: NCE) accelerating the target ion in a collision cell set to 40. The obtained MS spectrum is shown in FIG. 14.

The above NMR spectra and the MS² measurement results show that 8-chloro-4-hydroxy[1]benzofuro[3,2-d]pyrimidine-2-di (or 8-chloro[1]benzofuro[3,2-d]pyrimidine-4(1H)one-2-d₁) was synthesized.

This application is based on Japanese Patent Application Serial No. 2024-060825 filed with Japan Patent Office on Apr. 4, 2024, the entire contents of which are hereby incorporated by reference.

What is claimed is:

1. A method for synthesizing an organic compound represented by General Formula (G1), comprising the step of causing a reaction between an organic compound represented by General Formula (G0), a transition metal catalyst, heavy water, and at least one of a hydrogen molecule and an H₂ generation source, 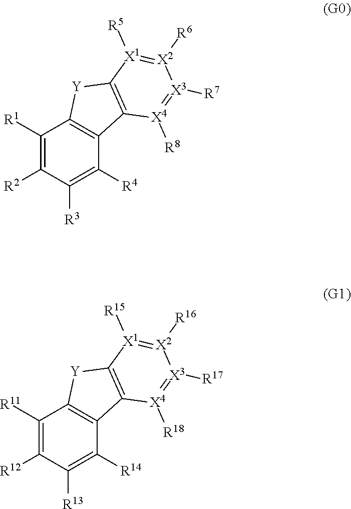
wherein Y represents oxygen or sulfur,
wherein each of R¹ to R⁴ and R¹¹ to R¹⁴ independently represents any one of hydrogen, a hydroxyl group, an organoboron group, a boronic acid, an organotin group, and a halogen,
wherein each of X¹ to X⁴ independently represents carbon or nitrogen and any one or two of X¹ to X⁴ represent nitrogen,
wherein each of R⁵ to R⁸ and R¹⁵ to R¹⁸ independently represents hydrogen, a hydroxyl group, an organoboron group, a boronic acid, an organotin group, or a halogen,
wherein when X¹ to X⁴ each represent nitrogen, R⁵ to R⁸ and R¹⁵ to R¹⁸ bonded to the nitrogen each represent a vacancy, and
wherein a sum of deuterium atoms substituting for R¹¹ to R¹⁸ is greater than a sum of deuterium atoms substituting for R¹ to R⁸.

2. The method for synthesizing an organic compound, according to claim 1, wherein the H₂ generation source is any of formic acid, ammonia borane, and methanol.

3. The method for synthesizing an organic compound, according to claim 1, wherein the H₂ generation source is 2-propanol.

4. The method for synthesizing an organic compound, according to claim 1, wherein the transition metal catalyst comprises a platinum group element.

5. The method for synthesizing an organic compound, according to claim 1, wherein each of X² and X⁴ represents nitrogen.

6. The method for synthesizing an organic compound, according to claim 1, wherein R¹⁷ represents deuterium.

7. The method for synthesizing an organic compound, according to claim 1, wherein each of R³ and R¹³ represents a halogen.

8. An organic compound represented by General Formula (G1), 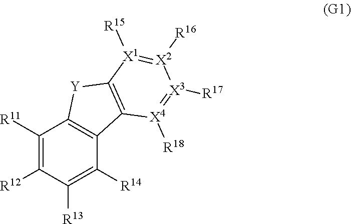
wherein each of R¹¹ to R¹⁴ independently represents any one of hydrogen, a hydroxyl group, and a halogen,
wherein each of X¹ to X⁴ independently represents carbon or nitrogen and any one or two of X¹ to X⁴ represent nitrogen,
wherein each of R¹⁵ to R¹⁸ independently represents hydrogen, a hydroxyl group, or a halogen,
wherein when X¹ to X⁴ each represent nitrogen, R¹⁵ to R¹⁸ bonded to the nitrogen each represent a vacancy,
wherein Y represents oxygen or sulfur, and
wherein at least one of R¹¹ to R¹⁸ represents deuterium.

9. An organic compound represented by at least one of Structural Formulae (100) and (100-1). 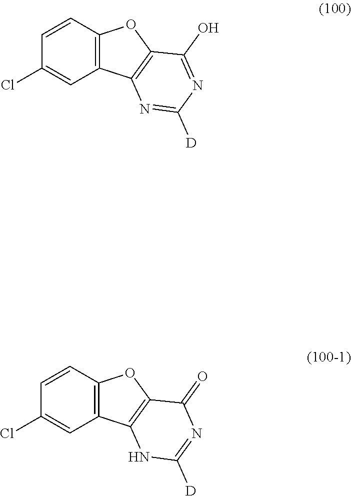 [SMILES: [2H]c1nc(O)c2oc3ccc(Cl)cc3c2n1 ; [2H]c1nc(=O)c2oc3ccc(Cl)cc3c2[nH]1]
